(19) United States
(12) Patent Application Publication
(10) Pub. No.: US 2008/0058400 A1
(43) Pub. Date: Mar. 6, 2008

(54) Skin external preparation

(75) Inventors: Chunying Yang, Sakado-shi (JP); Mario Aoki, Tokyo (JP); Takashi Usami, Tokyo (JP)

Correspondence Address: BIRCH STEWART KOLASCH & BIRCH, PO BOX 747, FALLS CHURCH, VA, 22040-0747, US

(73) Assignee: FUJIFILM CORPORATION

(21) Appl. No.: 11/892,113

(22) Filed: Aug. 20, 2007

(30) Foreign Application Priority Data
Aug. 29, 2006 (JP) P2006-231867
Aug. 29, 2006 (JP) P2006-231868

(51) Int. Cl. A61K 31/4164; A61P 17/00

(52) U.S. Cl. 514/399

(57) ABSTRACT

A skin external preparation includes at least arginine, aspartic acid, isoleucine, leucine, lysine, threonine, glycine, histidine, serine, valine, tyrosine, cysteine, phenylalanine, hydroxyproline and acylglutamine among amino acids, or salts thereof, and a skin external preparation includes: at least arginine, aspartic acid, isoleucine, leucine, lysine and threonine among amino acids, or salts thereof, and a hydrolyzed silk.

### BACKGROUND OF THE INVENTION

1. Field of the Invention

This invention relates to a skin external preparation which can be applied to medicines, quasi drugs, cosmetics (pharmaceutical preparations to be used for human and other animals) and the like various skin external preparations.

2. Description of the Related Art

Since the skin is covered with a horny layer which is a thin biological defense membrane, we can live in the dry air without parting with moisture, and this is because the horny layer is present in the skin contacting with the external moiety, namely in the epidermis. The horny layer prevents release of moisture from the body and, at the same time, carries out various regulations for maintaining normal conditions of the skin.

However, the skin is hardened by undergoing injuries by an external wound (e.g., a burn or an inflammation due to ultraviolet ray and the like various stimulative factors) and the like, and the horny layer therefore is cracked and peeled off, so that loss of a large quantity of moisture occurs in comparison with the skin of normal state.

In addition, on the contrary, as a cause of missing flexibility, extensibility and strength required by the horny layer, there is a case in which reduction of the skin moisture is the cause or concerned therein. A horny layer having a functional defect does not have a moisture-binding (keeping) ability sufficient for maintaining the horny layer itself flexible, so that a large quantity of moisture is passed and lost through the skin as a result on the contrary. In general, when the moisture content of the horny layer is reduced to 10% or less of its dry weight, the flexibility and softness of the skin so far maintained are lost, and it becomes hard and brittle, thus resulting in a so-called chapped, moist-less dry skin, so that it can be said that the moisture content of the skin horny layer is very important.

Accordingly, in order to compensate for this point, it is conventionally considered ideal as cosmetics to reproduce, on the skin, substances almost identical to the components on the skin surface, so that those which mainly employ moisture content and oil content as the typical components of the skin are frequently used. In addition, in recent years, a horny layer component called NMF (natural moisturizing factor) has been drawing attention as a third beauty component, and studies and developments on the amino acids and derivatives thereof as the NMF components have been energetically carried out and applied to cosmetics (moisture keeping, rinsing, ultraviolet ray absorption, antibacterial action and the like) (JP-A-61-289016 and JP-A-6-279227 and the like).

Since oligopeptides obtained by hydrolyzing natural proteins have a chemical structure similar to that of the natural peptide in the epidermis, they have been broadly used as a moisture keeping agent and the like. For example, hydrolyzed collagen, hydrolyzed silk and the like can be cited. However, their sufficient examinations have not been made as materials, in combination with amino acids, for improving skin permeability of amino acids and delivering them into inside of the epidermis.

On the other hand, cell activators and the like have also been proposed in many ways for the purpose of keeping the skin healthy. For example, it is known that the aforementioned amino acids have the cell activation action, and in addition to this, hydroxyproline and the like collagen related amino acid derivatives (JP-A-61-289016), N-acyl amino acid and the like various derivatives (JP-T-2002-508308 (the tem “JP-T” as used herein means a published Japanese translation of a PCT patent application)) have been provided.

However, when the additive agent is an amino acid for example, there are many kinds of amino acid and derivatives thereof, so that there remain problems regarding relationship between the combination of amino aids or amino acid derivatives and the efficacy thereof in the skin cells and their optimum formulation and combination determined by taking their permeation into the skin into consideration.

### SUMMARY OF THE INVENTION

As described in the above, various methods for using amino acids and derivatives thereof in the field of cosmetics, but there still is room for the improvement regarding combination and formulation of amino acids by taking their permeation into the skin into consideration.

Thus, the present inventors have conducted intensive studies on the additives regarding their formulation which shows cell activation action, has superior moisture keeping ability and is useful in medicines, quasi drugs and cosmetics.

The invention provides a skin external preparation which has the moisture keeping action and cell activation action and shows a permeating feeling.

The aforementioned object has been achieved by the following (1) to (18).

(1) A skin external preparation, which comprises at least arginine, aspartic acid, isoleucine, leucine, lysine, threonine, glycine, histidine, serine, valine, tyrosine, cysteine, phenylalanine, hydroxyproline and acylglutamine among amino acids, or salts thereof.

(2) The skin external preparation as described in (1) above,

wherein, with respect to adding amounts of the amino acids or the salts thereof, an adding amount of arginine is from 20 to 400μ% by weight, an adding amount of aspartic acid is from 3 to 60μ% by weight, an adding amount of isoleucine is from 30 to 600μ% by weight, an adding amount of leucine is from 30 to 600μ% by weight, an adding amount of lysine is from 30 to 600μ% by weight, an adding amount of threonine is from 25 to 500μ% by weight, an adding amount of glycine is from 6 to 120μ% by weight, an adding amount of histidine is from 8 to 160μ% by weight, an adding amount of serine is from 8 to 160μ% by weight, an adding amount of valine is from 30 to 500μ% by weight, an adding amount of tyrosine is from 0.08 to 20μ% by weight, an adding amount of cysteine is from 15 to 300μ% by weight, an adding amount of phenylalanine is from 0.12 to 20μ% by weight and an adding amount of hydroxyproline is from 5 to 150μ% by weight.

(3) The skin external preparation as described in (1) or (2) above,

wherein the acylglutamine is acetylglutamine.

(4) The skin external preparation as described in (3) above,

wherein an adding amount of the acetylglutamine is from 10 to 800μ% by weight.

(5) The skin external preparation as described in any of (1) to (4) above, which further comprises a vitamin B.

(6) The skin external preparation as described in (5) above,

wherein the vitamin B is vitamin B₁ or vitamin B₆.

(7) The skin external preparation as described in any of (1) to (6) above, which further comprises hyaluronic acid or a salt thereof.

(8) The skin external preparation as described in any of (1) to (7) above, which is a cosmetic or a quasi drug.

(9) The skin external preparation as described in (8) above, which is a face lotion, an emulsion, a cream, a hair tonic or a pack.

(10) A skin external preparation, which comprises:

at least arginine, aspartic acid, isoleucine, leucine, lysine and threonine among amino acids, or salts thereof; and

a hydrolyzed silk.

(11) The skin external preparation as described in (10) above,

wherein, with respect to adding amounts of the amino acids or the salts thereof, an adding amount of arginine is from 20 to 400μ% by weight, an adding amount of aspartic acid is from 3 to 60μ% by weight, an adding amount of isoleucine is from 30 to 600μ% by weight, an adding amount of leucine is from 30 to 600μ% by weight, an adding amount of lysine is from 30 to 600μ% by weight and an adding amount of threonine is from 25 to 500μ% by weight.

(12) The skin external preparation as described in (10) or (11) above,

wherein an adding amount of the hydrolyzed silk is from 5 to 3,000μ% by weight.

(13) The skin external preparation as described in any of (10) to (12) above, which further comprises at least one of glycine, histidine, serine, valine, tyrosine, cysteine, phenylalanine and salts thereof, among amino acids.

(14) The skin external preparation as described in any of (10) to (13) above, which further comprises a vitamin B.

(15) The skin external preparation as described in (14) above,

wherein the vitamin B is vitamin B₁ or vitamin B₆.

(16) The skin external preparation as described in any of (10) to (15) above, which further comprises hyaluronic acid or a salt thereof.

(17) The skin external preparation as described in any of (10) to (16) above, which is a cosmetic or a quasi drug.

(18) The skin external preparation as described in (17) above, which is a face lotion, an emulsion, a cream, a hair tonic or a pack.

### DETAILED DESCRIPTION OF THE INVENTION

The amino acids to be used as essential in the first embodiment of the invention are arginine, aspartic acid, isoleucine, leucine, lysine, threonine, glycine, histidine, serine, valine, tyrosine, cysteine and phenylalanine or salts thereof. They may be either optically active substances or racemic modifications, but it is particularly desirable that all of them are in L form.

Adding amounts of the amino acids to be used as essential in the first embodiment of the invention are described in detail.

Adding amount of arginine is preferably from 20 to 400μ% by weight, more preferably from 40 to 200μ% by weight, most preferably from 60 to 120μ% by weight.

Adding amount of aspartic acid is preferably from 3 to 60μ% by weight, more preferably from 8 to 30μ% by weight, most preferably from 12 to 20μ% by weight.

Adding amount of isoleucine is preferably from 30 to 600μ% by weight, more preferably from 50 to 300μ% by weight, most preferably from 80 to 200μ% by weight.

Adding amount of leucine is preferably from 30 to 600μ% by weight, more preferably from 50 to 300μ% by weight, most preferably from 80 to 200μ% by weight.

Adding amount of lysine is preferably from 30 to 600μ% by weight, more preferably from 50 to 300μ% by weight, most preferably from 80 to 200μ% by weight.

Adding amount of threonine is preferably from 25 to 250μ% by weight, more preferably from 40 to 150μ% by weight, most preferably from 60 to 120μ% by weight.

Adding amount of glycine is preferably from 6 to 120μ% by weight, more preferably from 16 to 60μ% by weight, most preferably from 20 to 40μ% by weight.

Adding amount of histidine is preferably from 8 to 160μ% by weight, more preferably from 20 to 90μ% by weight, most preferably from 30 to 60μ% by weight.

Adding amount of serine is preferably from 8 to 160μ% by weight, more preferably from 20 to 90μ% by weight, most preferably from 30 to 60μ% by weight.

Adding amount of valine is preferably from 30 to 500μ% by weight, more preferably from 50 to 200μ% by weight, most preferably from 70 to 150μ% by weight.

Adding amount of tyrosine is preferably from 0.08 to 20μ% by weight, more preferably from 0.15 to 15μ% by weight, most preferably from 0.3 to 12μ% by weight.

Adding amount of cysteine is preferably from 15 to 300μ% by weight, more preferably from 30 to 150μ% by weight, most preferably from 40 to 80μ% by weight.

Adding amount of phenylalanine is preferably from 0.12 to 20μ% by weight, more preferably from 0.15 to 10μ% by weight, most preferably from 0.3 to 1μ% by weight.

Next, the hydroxyproline to be used in the first embodiment of the invention is described.

Hydroxyproline may be either an optically active substance or a racemic modification, but L form is particularly desirable. The hydroxy group may be cis or trans. Adding amount of hydroxyproline is preferably from 5 to 150μ% by weight, more preferably from 10 to 90μ% by weight, most preferably from 15 to 60μ% by weight.

The acylglutamine to be used in the first embodiment of the invention may be either an optically active substance or a racemic modification, but L form is particularly desirable. The acyl group is preferably an acyl group having from 2 to 8 carbon atoms, more preferably an acyl group having from 2 to 6 carbon atoms, most preferably acetyl group having from 2 carbon atoms. The acylglutamine is not particularly limited, but acetylglutamine is preferable. Adding amount of the acylglutamine is preferably from 10 to 800μ% by weight, more preferably from 20 to 400μ% by weight, most preferably from 40 to 200μ% by weight.

The amino acids to be used as essential in the second embodiment of the invention are arginine, aspartic acid, isoleucine, leucine, lysine and threonine, which may be either optically active substances or racemic modifications, but it is particularly desirable that all of them are in L form.

Adding amounts of the amino acids to be used as essential in the second embodiment of the invention are described in detail.

Adding amount of arginine is preferably from 20 to 400μ% by weight, more preferably from 40 to 200μ% by weight, most preferably from 60 to 120μ% by weight.

Adding amount of aspartic acid is preferably from 3 to 60μ% by weight, more preferably from 8 to 30μ% by weight, most preferably from 12 to 20μ% by weight.

Adding amount of isoleucine is preferably from 30 to 600μ% by weight, more preferably from 50 to 300μ% by weight, most preferably from 80 to 200μ% by weight.

Adding amount of leucine is preferably from 30 to 600μ% by weight, more preferably from 50 to 300μ% by weight, most preferably from 80 to 200μ% by weight.

Adding amount of lysine is preferably from 30 to 600μ% by weight, more preferably from 50 to 300μ% by weight, most preferably from 80 to 200μ% by weight.

Adding amount of threonine is preferably from 25 to 250μ% by weight, more preferably from 40 to 150μ% by weight, most preferably from 60 to 120μ% by weight.

The hydrolyzed silk to be used in the second embodiment of the invention is a product obtained by hydrolyzing silk protein with an acid or an enzyme or by other method, but the hydrolyzing method is not particularly limited. Adding amount of the hydrolyzed silk is preferably from 5 to 3,000μ% by weight, more preferably from 10 to 1,000μ% by weight, most preferably from 20 to 200μ% by weight.

Next, the amino acids to be used by further adding to the essential amino acids in the second embodiment of the invention are described. The amino acids to be used by their further addition are glycine, histidine, serine, valine, tyrosine, cysteine and phenylalanine, which may be may be either optically active substances or racemic modifications, but it is particularly desirable that all of them are in L form. It is desirable that at least 1 species or more of these amino acids are added, but the addition of 3 species or more is further desirable, and the addition of all of the 8 species is most desirable.

Next, adding amounts of the amino acids to be used by their further addition in the second embodiment of the invention are described.

Adding amount of glycine is preferably from 6 to 120μ% by weight, more preferably from 16 to 60μ% by weight, most preferably from 20 to 40μ% by weight.

Adding amount of histidine is preferably from 8 to 160μ% by weight, more preferably from 20 to 90μ% by weight, most preferably from 30 to 60μ% by weight.

Adding amount of serine is preferably from 8 to 160μ% by weight, more preferably from 20 to 90μ% by weight, most preferably from 30 to 60μ% by weight.

Adding amount of threonine is preferably from 30 to 500μ% by weight, more preferably from 50 to 200μ% by weight, most preferably from 70 to 150μ% by weight.

Adding amount of valine is preferably from 30 to 500μ% by weight, more preferably from 50 to 200μ% by weight, most preferably from 70 to 150μ% by weight.

Adding amount of tyrosine is preferably from 0.08 to 1.6μ% by weight, more preferably from 0.2 to 0.9μ% by weight, most preferably from 0.3 to 0.6μ% by weight.

Adding amount of cysteine is preferably from 15 to 300μ% by weight, more preferably from 30 to 150μ% by weight, most preferably from 40 to 80μ% by weight.

Adding amount of phenylalanine is preferably from 0.12 to 2.4μ% by weight, more preferably from 0.2 to 1.5μ% by weight, most preferably from 0.3 to 1.0μ% by weight.

Next, the additives to be used commonly in the first and second embodiment of the invention are described.

Vitamins have been used in various skin external preparations because of their coenzyme activities, but when used in combination with the composition of the invention, vitamins belonging to the vitamin B group are particularly desirable. Illustrative examples of the vitamin B group include vitamin B₁, vitamin B₂, vitamin B₃, vitamin B₄, vitamin B₅, vitamin B₆, vitamin B₁₂, vitamin B₁₃, vitamin B₁₅, vitamin B₁₇, vitamin B_{T}, vitamin Bₓ and vitamin B_{c}, of which vitamin B₁ concerned in the sugar metabolism, vitamin B₂ concerned in the lipid metabolism and vitamin B₆ concerned in the protein metabolism are preferable, and vitamin B₁ and vitamin B₆ are more preferable.

Adding amount of the vitamin B group to be used in the invention is preferably from 0.8 to 16μ% by weight, more preferably from 2 to 9μ% by weight, most preferably from 3 to 6μ% by weight.

According to the invention, it is particularly desirable to add hyaluronic acid for the purpose of keeping flexibility of the skin, and the hyaluronic acid may be sodium salt, potassium salt or free acid. When the skin external preparation of the invention is a cosmetic, addition of hyaluronic acid is particularly desirable. Adding amount of hyaluronic acid to be used in the skin external preparation is preferably from 150 to 3,000μ% by weight, more preferably from 300 to 1,500μ% by weight, most preferably from 400 to 800μ% by weight.

The skin external preparation of the invention can be concomitantly used with various drug effect agents by adding them thereto. Examples of such drug effect agents include an active oxygen removing agent, an antioxidant, a cell activator, an anti-inflammatory agent, a tyrosinase activity inhibitor, a UV absorbing agent and a moisture keeping agent. As the illustrative drug effect agents, respective agents shown in the following can be exemplified, though not limited thereto as a matter of course.

The active oxygen removing agent or antioxidant which can be used in the invention has no particular limitation, but a substance extracted or purified from a natural material is desirable rather than a substance obtained by chemical synthesis.

As the active oxygen removing agent, for example, β-carotene, α-carotene, lycopene, lutein, astaxanthin, capsanthin, fucoxanthin, heteroxanthin, loroxanthin, luteoxanthin, lycophyl, lycoxanthin, neochrome, neoxanthin, rhodopin, rhodopinal, rhodopinol, rhodovibrin, trollixanthin, xanthophyll, zeaxanthin and the like carotenoids, superoxide dismutase, mannitol, rutin and a derivative thereof, bilirubin, cholesterol, tryptophan, histidine, quercetin, quercitrin, catechin, catechin derivatives, gallic acid and a derivative thereof, glutathione and a derivative thereof and a salt thereof, and Scutellaria root extract, Ginkgo biloba extract, Millettia reticulata extract, Howthorn Fruit extract, Rosa rugosa extract, Saxifraga stolonifera extract, Melissa officinalis extract, Geranium thunbergii extract, Moutan Bark extract, parsley extract, Potentilla tormentilla extract, Momordica grosvenori extract, Yashajitsu extract, Lycii cortex extract and the like flavonoid-containing plant extracts can be cited.

As the antioxidant, for example, retinol palmitate, retinol acetate and the like retinol and a derivative thereof, retinal and a derivative thereof, dehydroretinal and the like vitamin A group; thiamine hydrochloride, thiamin sulfate and the like thiamines, riboflavin, pyridoxine hydrochloride, pyridoxine dioctanoate and the like pyridoxines, flavin adenine nucleotide, cyanocobalamin, folic acids, nicotinic acid amide, benzyl nicotinate and the like nicotinic acids, calcium pantothenate, D-pantothenyl alcohol, pantothenyl ethyl ether, acetyl pantothenyl ethyl ether and the like pantothenic acids, choline, inositol and the like vitamin B group; L-ascorbic acid, L-ascorbyl palmitate, L-ascorbyl dipalmitate, L-ascorbyl isopalmitate, L-ascorbyl diisopalmitate, L-ascorbyl tetraispalmitate, L-ascorbyl stearate, L-ascorbyl distearate, L-ascorbyl isostearate, L-ascorbyl diisostearate, L-ascorbyl myristate, L-ascorbyl dimyristate, L-ascorbyl isomyristate, L-ascorbyl diisomyristate, L-ascorbyl oleate, L-ascorbyl dioleate, L-ascorbyl 2-ethylhexanoate, L-ascorbic acid phosphoric acid ester sodium, L-ascorbic acid phosphoric acid ester potassium, L-ascorbic acid phosphoric acid ester magnesium, L-ascorbic acid phosphoric acid ester calcium, L-ascorbic acid phosphoric acid ester aluminum, L-ascorbic acid sulfuric acid ester sodium, L-ascorbic acid sulfuric acid ester potassium, L-ascorbic acid sulfuric acid ester magnesium, L-ascorbic acid sulfuric acid ester calcium, L-ascorbic acid sulfuric acid ester aluminum, sodium L-ascorbate, potassium L-ascorbate, magnesium L-ascorbate, calcium L-ascorbate, aluminum L-ascorbate and the like ascorbic acid and a derivative thereof and a salt thereof as the vitamin C group; ergocalciferol, cholecalciferol, dihydroxystanal and the like vitamin D group; d1-α(β, γ)-tocopherol, d1-α-tocopherol acetate, d1-α-tocopherol nicotinate, d1-α-tocopherol linoleate, d1-α-tocopherol succinate and the like tocopherol and a derivative thereof and a salt thereof, ubiquinone and the like vitamin E group; dibutylhydroxytoluene and dibutylhydroxyanisole and the like can be cited.

Preferred among the aforementioned active oxygen removing agents and antioxidants include mannitol, β-carotene, astaxanthin, rutin and derivatives thereof, Ginkgo biloba extract, Scutellaria root extract, Millettia reticulata extract, Saxifraga stolonifera extract, Melissa officinalis extract, Geranium thunbergii extract, Eijitsu extract, vitamin C group and derivatives thereof and salts thereof, and vitamin E and derivatives thereof and salts thereof, of which more preferred are β-carotene, astaxanthin, Ginkgo biloba extract, vitamin C group and derivatives thereof and salts thereof and vitamin E and derivatives thereof and salts thereof.

Concentration of the active oxygen removing agent or antioxidant in the skin external preparation of the invention is preferably from 0.00001 to 50% by weight, more preferably from 0.0001 to 30% by weight. However, in the case of the use of a plant extract, there is no problem when its dry solid content is within the aforementioned range. In addition, the active oxygen removing agent or antioxidant can be used alone or by combining two or more species.

### (Cell Activator)

As the cell activator, for example, deoxyribonucleic acid and a salt thereof, adenosine triphosphate, adenosine monophosphate and the like adenylic acid derivatives and salts thereof, guanine, xanthine and derivatives and salts thereof and the like nucleic acid-related substances; serum deproteinization extract, spleen extract, placenta extract, cockscomb extract, royal jelly and the like animal-derived extracts; yeast extract, lactic acid fermentation extract, bifidobacterium extract, Fomes japonicus extract and the like microorganism-derived extracts; carrot extract, Swertia japonica extract, rosemary extract, phellodendron bark extract, garlic extract, hinokitiol, cepharanthin and the like plant-derived extracts; α- or γ-linolenic acid, eicosapentaenoic acid and a derivative thereof, succinic acid and a derivative thereof and a salt thereof, estradiol and a derivative thereof and a salt thereof, lactic acid, glycolic acid, citric acid, malic acid, salicylic acid and the like α-hydroxy acids and a derivative thereof and a salt thereof and the like can be cited.

Among these cell activators, particularly preferred are deoxyribonucleic acid and a salt thereof, adenosine triphosphate and a salt thereof, serum deproteinization extract, placenta extract, yeast extract, lactic acid fermentation extract, carrot extract, hinokitiol and succinic acid and a derivative thereof and a salt thereof.

Concentration of the cell activator in the skin external preparation of the invention is preferably from 0.0001 to 5% by weight, more preferably from 0.001 to 3% by weight. However, in the case of the use of a plant extract, there is no problem when its dry solid content is within the aforementioned range. In addition, the cell activator can be used alone or by combining two or more species.

### (Anti-Inflammatory Agent)

As the anti-inflammatory agent, glycyrrhizic acid, glycyrrhetic acid, mefenamic acid, phenylbutazone, indometacin, ibuprofen, ketoprofen, allantoin, guaiazulene and derivatives thereof and salts thereof, ε-aminocaproic acid, zinc oxide, diclofenac sodium, aloe extract, salvia extract, arnica extract, Chamomillae Flos extract, white birch extract, Hypericum erectum Thunberg extract, Eucalyptus extract, Sapindus mukurossi Gaertn. extract and the like can be exemplified.

Among these anti-inflammatory agents, particularly preferred are glycyrrhizic acid, glycyrrhetic acid, guaiazulene and derivatives thereof and salts thereof, ε-aminocaproic acid, aloe extract and Chamomillae Flos extract.

Concentration of the anti-inflammatory agent in the composition is generally from 0.0001 to 1%, preferably from 0.01 to 0.5% by weight. However, in the case of the use of a plant extract, there is no problem when its dry solid content is within the aforementioned range. In addition, the anti-inflammatory agent can be used alone or by combining two or more species.

### (Tyrosinase Activity Inhibitor)

As the tyrosinase activity inhibitor, cysteine and a derivative thereof (e.g., N,N′-diacetylcystinedimethyl or the like) and a salt thereof, Inula britannica extract, Millettia reticulata extract, Cassia nomane extract, Mori Cortex extract, Angelicae Radix extract, Polygonum bistorta extract, Clara extract, Howthorn Fruit extract, lily bulb extract, hops extract, Rosa multiflora extract, Coicis Semen extract and the like can be exemplified.

Concentration of the tyrosinase activity inhibitor is preferably from 0.0001 to 2%, particularly preferably from 0.001 to 0.5% by weight. However, in the case of the use of a plant extract, there is no problem when its dry solid content is within the aforementioned range. In addition, these can be used alone or by combining two or more species.

### (Moisture Keeping Agent)

As the moisture keeping agent, not only urea and the like synthetic compounds but also amino acids, pyrrolidonecarboxylic acid, lactic acid salts and the like low molecular compounds known as natural moisture keeping agents can be used. In addition, mucopolysaccharides and/or protein conventionally formulated in cosmetics can be used.

Among them, the amino acids described in “Development of New Practical Cosmetics (written in Japanese)”, edited by M. Suzuki, published by CMC on September, 2004, page 16 to page 19, can be exemplified as the amino acids.

Also, as the mucopolysaccharides, hyaluronic acid, chondroitin sulfate, dermatan sulfate, heparan sulfate, heparin and keratan sulfate and salts thereof can for example be cited, and hyaluronic acid, chondroitin sulfate and salts thereof can be used particularly preferably.

In addition, as the protein, collagen, elastin, keratin and derivatives thereof and salts thereof can for example be cited, of which collagen is particularly preferable. Origins of these respective components have no particular limitation, and they may be any one of animal origins, microbial origins and synthetic products. The extraction method and purification method in the case of natural origins also have no particular limitation.

These moisture keeping components can be used alone or by simultaneously adding two or more species as occasion demands.

In addition, in general, blending amount of the moisture keeping agent is preferably from 0.0001 to 5%, more preferably from 0.001 to 3%, though it may vary depending on the combination of its components.

The composition containing the amino acids of the invention or their salts, and the composition containing the amino acids of the invention or their salts and hydrolyzed silk can be prepared in accordance with the usual way, by blending with various types of bases known as general compositions for external use. That is, oils (natural animal or plant oil and fat, semi-synthetic oil and fat, hydrocarbon oil, higher fatty acid, ester oil, silicone oil, fluorine base oils and the like), gelling agents, metallic soap, surfactants (anionic, cationic, ampholytic), powders (inorganic powder, organic powder, pigment and the like), alcohols (higher alcohol, polyhydric alcohol, sterol and the like), water-soluble high polymers (animal or plant system, microbial system, synthetic system), coat forming agents, resins, antiseptics, antibacterial agents, perfumes, essential oils, salts, water (purified water, hot spring water and deep water), pH adjusting agents, refrigerants, chapped skin improving agents, blood circulation accelerating agents, skin astringents, antiseborrheic agents, amino acids, vitamins, nucleic acid-related substances, enzymes, hormones, inclusion compounds, plant extracts, animal- and microbial origin extracts, ultraviolet ray scattering agents and the like can be added.

Regarding the amino acids and hydrolyzed silk to be used in the invention, and vitamin B group and hyaluronic acid which are used by further adding thereto, their using amounts exceeding the upper limit of the adding amounts of the invention do not produce negative physiological effects, but their positive effects are saturated at around the upper limits shown by the invention so that a profit of increasing their adding amounts cannot be obtained. In addition, their addition exceeding the upper limits of the invention may sometimes result in the worsening of used feeling such as stickiness.

### EXAMPLES

The following describes the invention in detail based on examples, but the invention is not limited thereto as a matter of course.

Effect of the amino acid content in a medium on the growth of epidermal keratinocytes

a) Culturing of Human Epidermal Keratinocytes

Human normal epidermal keratinocytes (Sanko Junyaku) were used as the cells. An MCDB 153 medium (Kohjin Bio) supplemented with 50 mg/l of BPE (bovine pituitary gland extract, Kohjin Bio), 5 mg/l of insulin (SIGMA), 0.1 mM ethanolamine (SIGMA), 0.1 mM phosphoethanolamine (SIGMA) and 0.00001 mg EGF (epithelial cell growth factor, SIGMA) as growth factors was used as the medium. The cells were pre-cultured at 37° C., under an atmosphere of 5% (v/v) CO₂ until they became a semi-confluent state.

b) Preparation of Human Epidermal Keratinocytes

The keratinocytes cultured under a growing state in a T 75 flask were used. After removing the medium by suction, 5 ml of 0.25% trypsin solution was added thereto and incubated at room temperature for 10 minutes. A 10 ml portion of the medium was added thereto, and the cells were peeled off and recovered by pipetting. The thus obtained cell suspension was centrifuged at 1,000 rpm for 5 minutes, and the supernatant was discarded. The thus obtained cell pellet was suspended in 10 ml of the medium and centrifuged at 1,000 rpm for 5 minutes, and the supernatant was discarded. The thus obtained cell pellet was again suspended in 10 ml of the medium and centrifuged at 1,000 rpm for 5 minutes, and the supernatant was discarded. The thus obtained cell pellet was suspended in 2 ml of the medium, and the number of cells was counted.

c) Growth of Human Epidermal Keratinocytes on Various Amino Acid Concentrations

In order to verify optimum blending amount of each amino acid, the MCDB 153 medium was prepared by changing concentration of each amino acid of interest alone. As growth factors, 50 mg/l of BPE (bovine pituitary gland extract), 5 mg/l of insulin, 0.1 mM ethanolamine, 0.1 mM phosphoethanolamine and 0.00001 mg EGF (epithelial cell growth factor) were added thereto. The aforementioned cells were mixed with 1 ml of this medium at a density of 3×10⁴ cells, inoculated onto a 24 well plate and cultured at 37° C. for 5 days under an atmosphere of 5% (v/v) CO₂, and then the number of cells was counted.

### Inventive Example 1-1

### Effect of Amino Acids on the Growth of Human Epidermal Keratinocytes

By reducing only one species among 20 amino acid species (valine, leucine, isoleucine, alanine, arginine, glutamine, lysine, aspartic acid, glutamic acid, cysteine, threonine, methionine, histidine, phenylalanine, tyrosine, tryptophan, asparagine, glycine and serine; mfd. by Wako Pure Chemical Industries) to 10% by weight of the corresponding general MCDB 153 medium concentration, a specific amino acid deficient medium was prepared and compared with a non-deficient medium. Relative ratios to the case of the non-deficient medium in which the number of cells was regarded as 100 are shown in the following Table 1-1.

TABLE 1-1
Amino acid deficient medium culturing test

| Component | Relative ratio |
|---|---|
| Alanine | 14 |
| Arginine | 8 |
| Asparagine | 13 |
| Aspartic acid | 6 |
| Cysteine | 9 |
| Glutamine | 16 |
| Glutamic acid | 15 |
| Glycine | 9 |
| Histidine | 7 |
| Isoleucine | 7 |
| Leucine | 6 |
| Lysine | 9 |
| Methionine | 12 |
| Phenylalanine | 4 |
| Proline | 13 |
| Serine | 6 |
| Threonine | 8 |
| Tryptophan | 15 |
| Tyrosine | 6 |
| Valine | 9 |
| Non-deficient | 100 |

It can be seen from Table 1-1 that the growth was suppressed particularly by the media deficient in the amino acids of the invention, arginine, aspartic acid, isoleucine, leucine, lysine, threonine, glycine, histidine, serine, valine, tyrosine, cysteine and phenylalanine, and therefore that the aforementioned amino acids are particularly necessary for the growth of human epidermal keratinocytes.

### Inventive Example 1-2

### Effects of Amino Acids and Additive Agents on the Growth of Human Epidermal Keratinocytes

Test medium compositions were prepared by adding amino acids to the MCDB 153 medium in such a manner that respective amino acid compositions became the following Tables 1-2 to 1-23.

In this connection, the test medium of Table 1-19 is a medium similar to the conventionally known composition shown in the examples of JP-A-61-289016.

Relative ratios, when the number of cells in the normal MCDB 153 medium with no addition of amino acids was regarded as 100, are shown in the following Tables 1-24 to 1-27.

TABLE 1-2
Test medium

| | Inventive | Inventive | Inventive | Inventive |
|---|---|---|---|---|
| | composition | composition | composition | composition |
| | 1-2-1 | 1-2-2 | 1-2-3 | 1-2-4 |
| | Concentration | Concentration | Concentration | Concentration |
| Components | (μg/ml) | (μg/ml) | (μg/ml) | (μg/ml) |
| Arginine | 80 | 80 | 160 | 400 |
| Aspartic acid | 15 | 15 | 30 | 60 |
| Cysteine | 50 | 50 | 100 | 200 |
| Glycine | 30 | 30 | 60 | 100 |
| Histidine | 40 | 40 | 80 | 150 |
| Isoleucine | 100 | 100 | 200 | 500 |
| Leucine | 100 | 100 | 200 | 500 |
| Lysine | 100 | 100 | 200 | 500 |
| Phenylalanine | 10 | 10 | 20 | 20 |
| Serine | 40 | 40 | 80 | 150 |
| Threonine | 90 | 90 | 180 | 400 |
| Tyrosine | 10 | 10 | 15 | 20 |
| Valine | 90 | 90 | 180 | 400 |
| Acetylglutamine | 100 | 100 | 200 | 400 |
| Hydroxyproline | 30 | 30 | 60 | 120 |
| Vitamin B₆ | 0 | 4 | 4 | 4 |
| Vitamin B₁ | 0 | 4 | 4 | 4 |

TABLE 1-3
Test medium

| | Inventive | Inventive | Inventive | Inventive |
|---|---|---|---|---|
| | composition | composition | composition | composition |
| | 1-2-5 | 1-2-6 | 1-2-7 | 1-2-8 |
| | Concentration | Concentration | Concentration | Concentration |
| Components | (μg/ml) | (μg/ml) | (μg/ml) | (μg/ml) |
| Arginine | 30 | 300 | 80 | 80 |
| Aspartic acid | 15 | 15 | 5 | 60 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-4
Test medium

| | Inventive | Inventive | Inventive | Inventive |
|---|---|---|---|---|
| | composition | composition | composition | composition |
| | 1-2-9 | 1-2-10 | 1-2-11 | 1-2-12 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 250 | 50 | 50 |
| Glycine | 30 | 30 | 8 | 100 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-5
Test medium

| | Inventive | Inventive | Inventive | Inventive |
|---|---|---|---|---|
| | composition | composition | composition | composition |
| | 1-2-13 | 1-2-14 | 1-2-15 | 1-2-16 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 10 | 150 | 40 | 40 |
| Isoleucine | 100 | 100 | 50 | 500 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-6
Test medium

| | Inventive | Inventive | Inventive | Inventive |
|---|---|---|---|---|
| | composition | composition | composition | composition |
| | 1-2-17 | 1-2-18 | 1-2-19 | 1-2-20 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 40 | 500 | 100 | 100 |
| Lysine | 100 | 100 | 50 | 500 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-7
Test medium

| | | Inventive | Inventive | Inventive |
|---|---|---|---|---|
| | Inventive composition | composition | composition | composition |
| | 1-2-21 | 1-2-22 | 1-2-23 | 1-2-24 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 0.15 | 15 | 10 | 10 |
| Serine | 40 | 40 | 10 | 150 |
| Threonine | 90 | 90 | 90 | 90 |
| Tyrosine | 10 | 10 | 10 | 10 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-8
Test medium

| | | Inventive | Inventive | Inventive |
|---|---|---|---|---|
| | Inventive composition | composition | composition | composition |
| | 1-2-25 | 1-2-26 | 1-2-27 | 1-2-28 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 40 | 400 | 90 | 90 |
| Tyrosine | 10 | 10 | 0.1 | 400 |
| Valine | 90 | 90 | 90 | 90 |
| Acetylglutamine | 100 | 100 | 100 | 100 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-9
Test medium

| | | Inventive | Inventive | Inventive |
|---|---|---|---|---|
| | Inventive composition | composition | composition | composition |
| | 1-2-29 | 1-2-30 | 1-2-31 | 1-2-32 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 40 | 400 | 90 | 90 |
| Acetylglutamine | 100 | 100 | 15 | 600 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-10
Test medium

| | Inventive | Inventive |
|---|---|---|
| | composition | composition |
| | 1-2-33 | 1-2-34 |
| | Concentration | Concentration |
| Components | (μg/ml) | (μg/ml) |
| Arginine | 80 | 80 |
| Aspartic acid | 15 | 15 |
| Cysteine | 50 | 50 |
| Glycine | 30 | 30 |
| Histidine | 40 | 40 |
| Isoleucine | 100 | 100 |
| Leucine | 100 | 100 |
| Lysine | 100 | 100 |
| Phenylalanine | 10 | 10 |
| Serine | 40 | 40 |
| Threonine | 90 | 90 |
| Tyrosine | 10 | 10 |
| Valine | 90 | 90 |
| Acetylglutamine | 100 | 100 |
| Hydroxyproline | 8 | 100 |
| Vitamin B₆ | 4 | 4 |
| Vitamin B₁ | 4 | 4 |

TABLE 1-11
Test medium

| | | Inventive | Inventive | Inventive |
|---|---|---|---|---|
| | Inventive composition | composition | composition | composition |
| | 1-2-35 | 1-2-36 | 1-2-37 | 1-2-38 |
| | Concentration | Concentration | Concentration | Concentration |
| Components | (μg/ml) | (μg/ml) | (μg/ml) | (μg/ml) |
| Arginine | 80 | 80 | 100 | 80 |
| Aspartic acid | 15 | 15 | 20 | 15 |
| Cysteine | 50 | 50 | 60 | 80 |
| Glycine | 30 | 30 | 30 | 40 |
| Histidine | 40 | 40 | 40 | 30 |
| Isoleucine | 100 | 100 | 200 | 150 |
| Leucine | 100 | 100 | 200 | 150 |
| Lysine | 100 | 100 | 200 | 200 |
| Phenylalanine | 10 | 10 | 0.5 | 0.6 |
| Serine | 40 | 40 | 40 | 60 |
| Threonine | 90 | 90 | 120 | 50 |
| Tyrosine | 10 | 10 | 12 | 10 |
| Valine | 90 | 90 | 150 | 120 |
| Acetylglutamine | 100 | 100 | 150 | 60 |
| Hydroxyproline | 30 | 30 | 50 | 150 |
| Vitamin B₆ | 2 | 6 | 4 | 4 |
| Vitamin B₁ | 2 | 6 | 4 | 4 |

TABLE 1-12
Test medium

| | Inventive | Inventive |
|---|---|---|
| | composition | composition |
| | 1-2-39 | 1-2-40 |
| | Concentration | Concentration |
| Components | (μg/ml) | (μg/ml) |
| Arginine | 300 | 80 |
| Aspartic acid | 40 | 15 |
| Cysteine | 200 | 60 |
| Glycine | 100 | 30 |
| Histidine | 150 | 40 |
| Isoleucine | 300 | 80 |
| Leucine | 400 | 120 |
| Lysine | 300 | 120 |
| Phenylalanine | 15 | 20 |
| Serine | 150 | 150 |
| Threonine | 200 | 100 |
| Tyrosine | 20 | 18 |
| Valine | 400 | 60 |
| Acetylglutamine | 400 | 150 |
| Hydroxyproline | 100 | 40 |
| Vitamin B₆ | 4 | 4 |
| Vitamin B₁ | 4 | 4 |

TABLE 1-13
Test medium

| | Inventive | Inventive |
|---|---|---|
| | composition | composition |
| | 1-2-41 | 1-2-42 |
| | Concentration | Concentration |
| Components | (μg/ml) | (μg/ml) |
| Arginine | 40 | 80 |
| Aspartic acid | 15 | 40 |
| Cysteine | 30 | 20 |
| Glycine | 50 | 80 |
| Histidine | 80 | 50 |
| Isoleucine | 150 | 80 |
| Leucine | 150 | 80 |
| Lysine | 100 | 120 |
| Phenylalanine | 0.5 | 1 |
| Serine | 20 | 50 |
| Threonine | 200 | 80 |
| Tyrosine | 1 | 0.3 |
| Valine | 80 | 120 |
| Acetylglutamine | 80 | 60 |
| Hydroxyproline | 40 | 20 |
| Vitamin B₆ | 4 | 4 |
| Vitamin B₁ | 4 | 4 |

TABLE 1-14
Test medium

| | | Inventive | Inventive | Inventive |
|---|---|---|---|---|
| | Inventive composition | composition | composition | composition |
| | 1-2-43 | 1-2-44 | 1-2-45 | 1-2-46 |
| | Concentration | Concentration | Concentration | Concentration |
| Components | (μg/ml) | (μg/ml) | (μg/ml) | (μg/ml) |
| Arginine | 40 | 30 | 80 | 400 |
| Aspartic acid | 15 | 15 | 15 | 60 |
| Cysteine | 50 | 30 | 60 | 20 |
| Glycine | 50 | 16 | 30 | 8 |
| Histidine | 40 | 20 | 50 | 10 |
| Isoleucine | 100 | 80 | 40 | 400 |
| Leucine | 100 | 80 | 40 | 400 |
| Lysine | 80 | 100 | 40 | 400 |
| Phenylalanine | 1 | 1 | 0.2 | 1 |
| Serine | 50 | 60 | 80 | 10 |
| Threonine | 150 | 60 | 200 | 30 |
| Tyrosine | 3 | 10 | 0.2 | 5 |
| Valine | 120 | 140 | 200 | 100 |
| Acetylglutamine | 40 | 100 | 500 | 800 |
| Hydroxyproline | 15 | 30 | 10 | 100 |
| Vitamin B₆ | 4 | 4 | 4 | 4 |
| Vitamin B₁ | 4 | 4 | 4 | 4 |

TABLE 1-15
Test medium

| | | Comparative | Comparative | Comparative |
|---|---|---|---|---|
| | Comparative composition | composition | composition | composition |
| | 1-2-1 | 1-2-2 | 1-2-3 | 1-2-4 |
| | Concentration | Concentration | Concentration | Concentration |
| Components | (μg/ml) | (μg/ml) | (μg/ml) | (μg/ml) |
| Arginine | 0 | 80 | 80 | 80 |
| Aspartic acid | 15 | 0 | 15 | 15 |
| Cysteine | 50 | 50 | 0 | 50 |
| Glycine | 30 | 30 | 30 | 0 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 40 | 400 | 90 | 90 |
| Acetylglutamine | 100 | 100 | 15 | 600 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-16
Test medium

| | | Comparative | Comparative | Comparative |
|---|---|---|---|---|
| | Comparative composition | composition | composition | composition |
| | 1-2-5 | 1-2-6 | 1-2-7 | 1-2-8 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 0 | 40 | 40 | 40 |
| Isoleucine | 100 | 0 | 100 | 100 |
| Leucine | 100 | 100 | 0 | 100 |
| Lysine | 100 | 100 | 100 | 0 |
| Phenylalanine | 10 | 10 | 10 | 10 |
| Serine | 40 | 40 | 40 | 40 |
| Threonine | 90 | 90 | 90 | 90 |
| Tyrosine | 10 | 10 | 10 | 10 |
| Valine | 40 | 400 | 90 | 90 |
| Acetylglutamine | 100 | 100 | 15 | 600 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-17
Test medium

| | | Comparative | Comparative | Comparative |
|---|---|---|---|---|
| | Comparative composition | composition | composition | composition |
| | 1-2-9 | 1-2-10 | 1-2-11 | 1-2-12 |
| | Concentration | Concentration | Concentration | Concentration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Cysteine | 50 | 50 | 50 | 50 |
| Glycine | 30 | 30 | 30 | 30 |
| Histidine | 40 | 40 | 40 | 40 |
| Isoleucine | 100 | 100 | 100 | 100 |
| Leucine | 100 | 100 | 100 | 100 |
| Lysine | 100 | 100 | 100 | 100 |
| Phenylalanine | 0 | 10 | 10 | 10 |
| Serine | 40 | 0 | 40 | 40 |
| Threonine | 90 | 90 | 0 | 90 |
| Tyrosine | 10 | 10 | 10 | 0 |
| Valine | 40 | 400 | 90 | 90 |
| Acetylglutamine | 100 | 100 | 15 | 600 |
| Hydroxyproline | 30 | 30 | 30 | 30 |
| Vitamin B₆ | 4 | 4 | 4 | 4 |
| Vitamin B₁ | 4 | 4 | 4 | 4 |

TABLE 1-18
Test medium

| | Comparative | Comparative | Comparative |
|---|---|---|---|
| | composition | composition | composition |
| | 1-2-13 | 1-2-14 | 1-2-15 |
| | Concentration | Concentration | Concentration |
| Components | (μg/ml) | (μg/ml) | (μg/ml) |
| Arginine | 80 | 80 | 80 |
| Aspartic acid | 15 | 15 | 15 |
| Cysteine | 50 | 50 | 50 |
| Glycine | 30 | 30 | 30 |
| Histidine | 40 | 40 | 40 |
| Isoleucine | 100 | 100 | 100 |
| Leucine | 100 | 100 | 100 |
| Lysine | 100 | 100 | 100 |
| Phenylalanine | 10 | 10 | 10 |
| Serine | 40 | 40 | 40 |
| Threonine | 90 | 90 | 90 |
| Tyrosine | 10 | 10 | 10 |
| Valine | 0 | 400 | 90 |
| Acetylglutamine | 100 | 0 | 15 |
| Hydroxyproline | 30 | 30 | 0 |
| Vitamin B₆ | 4 | 4 | 4 |
| Vitamin B₁ | 4 | 4 | 4 |

TABLE 1-19
Test medium

| | Comparative | Comparative | Comparative |
|---|---|---|---|
| | composition | composition | composition |
| | 1-2-16 | 1-2-17 | 1-2-18 |
| | Concentration | Concentration | Concentration |
| Components | (μg/ml) | (μg/ml) | (μg/ml) |
| Isoleucine | 50 | 25 | 0 |
| Tryptophan | 25 | 12.5 | 0 |
| Threonine | 50 | 25 | 0 |
| Valine | 50 | 25 | 100 |
| Phenylalanine | 25 | 12.5 | 0 |
| Methionine | 25 | 12.5 | 0 |
| Lysine | 75 | 37.5 | 100 |
| Leucine | 50 | 25 | 0 |
| Glutamine | 300 | 150 | 100 |
| Myo-inositol | 3.5 | 1.75 | 2 |
| Vitamin B₆ | 2 | 1 | 0 |
| Pantothenic acid | 2 | 1 | 0 |
| Nicotinamide | 2 | 1 | 1 |
| Glucose | 500 | 250 | 1000 |
| Succinic acid | 0.5 | 0.25 | 0 |

TABLE 1-20
Test medium

| | Comparative | Comparative | Comparative |
|---|---|---|---|
| | composition | composition | composition |
| | 1-2-19 | 1-2-20 | 1-2-21 |
| | Concentration | Concentration | Concentration |
| Components | (μg/ml) | (μg/ml) | (μg/ml) |
| Arginine | 10 | 10 | 100 |
| Aspartic acid | 2 | 30 | 20 |
| Cysteine | 10 | 10 | 10 |
| Glycine | 5 | 30 | 4 |
| Histidine | 50 | 5 | 10 |
| Isoleucine | 150 | 100 | 20 |
| Leucine | 150 | 30 | 20 |
| Lysine | 150 | 30 | 30 |
| Phenylalanine | 0.8 | 0.5 | 0.3 |
| Serine | 40 | 40 | 30 |
| Threonine | 100 | 80 | 20 |
| Tyrosine | 10 | 8 | 10 |
| Valine | 100 | 20 | 100 |
| Acetylglutamine | 100 | 5 | 5 |
| Hydroxyproline | 50 | 10 | 4 |
| Vitamin B₆ | 4 | 4 | 4 |
| Vitamin B₁ | 4 | 4 | 4 |

TABLE 1-21
Test medium

| | Comparative | Comparative | Comparative |
|---|---|---|---|
| | composition | composition | composition |
| | 1-2-22 | 1-2-23 | 1-2-24 |
| | Concentration | Concentration | Concentration |
| Components | (μg/ml) | (μg/ml) | (μg/ml) |
| Arginine | 80 | 80 | 80 |
| Aspartic acid | 15 | 15 | 5 |
| Cysteine | 60 | 60 | 20 |
| Glycine | 60 | 30 | 60 |
| Histidine | 80 | 50 | 5 |
| Isoleucine | 200 | 150 | 20 |
| Leucine | 200 | 150 | 150 |
| Lysine | 200 | 150 | 150 |
| Phenylalanine | 0.8 | 0.8 | 3 |
| Serine | 5 | 40 | 50 |
| Threonine | 20 | 80 | 100 |
| Tyrosine | 0.06 | 5 | 10 |
| Valine | 120 | 80 | 20 |
| Acetylglutamine | 5 | 5 | 5 |
| Hydroxyproline | 3 | 3 | 5 |
| Vitamin B₆ | 4 | 4 | 4 |
| Vitamin B₁ | 4 | 4 | 4 |

TABLE 1-22
Test medium

| | Comparative | Comparative | Comparative |
|---|---|---|---|
| | composition | composition | composition |
| | 1-2-25 | 1-2-26 | 1-2-27 |
| | Concentration | Concentration | Concentration |
| Components | (μg/ml) | (μg/ml) | (μg/ml) |
| Arginine | 40 | 20 | 400 |
| Aspartic acid | 15 | 3 | 60 |
| Cysteine | 10 | 15 | 5 |
| Glycine | 5 | 5 | 5 |
| Histidine | 6 | 8 | 5 |
| Isoleucine | 100 | 30 | 10 |
| Leucine | 100 | 600 | 20 |
| Lysine | 200 | 300 | 20 |
| Phenylalanine | 0.3 | 0.8 | 20 |
| Serine | 6 | 60 | 160 |
| Threonine | 80 | 250 | 250 |
| Tyrosine | 0.05 | 20 | 30 |
| Valine | 100 | 500 | 600 |
| Acetylglutamine | 8 | 60 | 8 |
| Hydroxyproline | 5 | 4 | 3 |
| Vitamin B₆ | 4 | 4 | 4 |
| Vitamin B₁ | 4 | 4 | 4 |

TABLE 1-23
Test medium

| | Comparative | Comparative | Comparative |
|---|---|---|---|
| | composition | composition | composition |
| | 1-2-28 | 1-2-29 | 1-2-30 |
| | Concentration | Concentration | Concentration |
| Components | (μg/ml) | (μg/ml) | (μg/ml) |
| Arginine | 10 | 15 | 500 |
| Aspartic acid | 2 | 10 | 100 |
| Cysteine | 10 | 10 | 500 |
| Glycine | 4 | 5 | 150 |
| Histidine | 160 | 200 | 200 |
| Isoleucine | 600 | 500 | 300 |
| Leucine | 200 | 100 | 300 |
| Lysine | 150 | 500 | 300 |
| Phenylalanine | 3 | 30 | 30 |
| Serine | 40 | 200 | 200 |
| Threonine | 20 | 150 | 300 |
| Tyrosine | 15 | 30 | 30 |
| Valine | 20 | 25 | 30 |
| Acetylglutamine | 5 | 8 | 800 |
| Hydroxyproline | 60 | 5 | 150 |
| Vitamin B₆ | 4 | 4 | 4 |
| Vitamin B₁ | 4 | 4 | 4 |

TABLE 1-24
Human epidermal keratinocyte growth accelerating effect

| Medium | Relative concentration |
|---|---|
| No addition | 100 |
| Inventive composition 1-2-1 | 109 |
| Inventive composition 1-2-2 | 112 |
| Inventive composition 1-2-3 | 116 |
| Inventive composition 1-2-4 | 118 |
| Inventive composition 1-2-5 | 104 |
| Inventive composition 1-2-6 | 110 |
| Inventive composition 1-2-7 | 103 |
| Inventive composition 1-2-8 | 111 |
| Inventive composition 1-2-9 | 105 |
| Inventive composition 1-2-10 | 114 |
| Inventive composition 1-2-11 | 103 |
| Inventive composition 1-2-12 | 108 |
| Inventive composition 1-2-13 | 104 |
| Inventive composition 1-2-14 | 111 |
| Inventive composition 1-2-15 | 103 |
| Inventive composition 1-2-16 | 112 |
| Inventive composition 1-2-17 | 104 |
| Inventive composition 1-2-18 | 110 |
| Inventive composition 1-2-19 | 103 |
| Inventive composition 1-2-20 | 112 |
| Inventive composition 1-2-21 | 104 |
| Inventive composition 1-2-22 | 113 |
| Inventive composition 1-2-23 | 104 |
| Inventive composition 1-2-24 | 113 |
| Inventive composition 1-2-25 | 105 |
| Inventive composition 1-2-26 | 111 |
| Inventive composition 1-2-27 | 103 |
| Inventive composition 1-2-28 | 112 |
| Inventive composition 1-2-29 | 104 |
| Inventive composition 1-2-30 | 109 |
| Inventive composition 1-2-31 | 103 |
| Inventive composition 1-2-32 | 112 |
| Inventive composition 1-2-33 | 104 |
| Inventive composition 1-2-34 | 111 |

TABLE 1-25
Human epidermal keratinocyte growth accelerating effect

| Medium | Relative concentration |
|---|---|
| No addition | 100 |
| Inventive composition 1-2-35 | 110 |
| Inventive composition 1-2-36 | 113 |
| Inventive composition 1-2-37 | 120 |
| Inventive composition 1-2-38 | 118 |
| Inventive composition 1-2-39 | 117 |
| Inventive composition 1-2-40 | 116 |
| Inventive composition 1-2-41 | 114 |
| Inventive composition 1-2-42 | 115 |
| Inventive composition 1-2-43 | 112 |
| Inventive composition 1-2-44 | 108 |
| Inventive composition 1-2-45 | 106 |
| Inventive composition 1-2-46 | 104 |

TABLE 1-26
Human epidermal keratinocyte growth accelerating effect

| | |
|---|---|
| Comparative composition 1-2-1 | 84 |
| Comparative composition 1-2-2 | 88 |
| Comparative composition 1-2-3 | 83 |
| Comparative composition 1-2-4 | 85 |
| Comparative composition 1-2-5 | 83 |
| Comparative composition 1-2-6 | 84 |
| Comparative composition 1-2-7 | 86 |
| Comparative composition 1-2-8 | 83 |
| Comparative composition 1-2-9 | 84 |
| Comparative composition 1-2-10 | 87 |
| Comparative composition 1-2-11 | 82 |
| Comparative composition 1-2-12 | 81 |
| Comparative composition 1-2-13 | 86 |
| Comparative composition 1-2-14 | 98 |
| Comparative composition 1-2-15 | 101 |
| Comparative composition 1-2-16 | 98 |
| Comparative composition 1-2-17 | 102 |
| Comparative composition 1-2-18 | 101 |

TABLE 1-27
Human epidermal keratinocyte growth accelerating effect

| | |
|---|---|
| Comparative composition 1-2-19 | 98 |
| Comparative composition 1-2-20 | 96 |
| Comparative composition 1-2-21 | 97 |
| Comparative composition 1-2-22 | 96 |
| Comparative composition 1-2-23 | 102 |
| Comparative composition 1-2-24 | 99 |
| Comparative composition 1-2-25 | 96 |
| Comparative composition 1-2-26 | 99 |
| Comparative composition 1-2-27 | 100 |
| Comparative composition 1-2-28 | 101 |
| Comparative composition 1-2-29 | 97 |
| Comparative composition 1-2-30 | 99 |

The inventive composition 1-2-1 accelerated growth of human epidermal keratinocyte, and the effect was increased by vitamin B₆ and vitamin B₁ (inventive composition 1-2-2). The acceleration effect became large as the amino acid concentration was increased, but the acceleration effect was not infinite and was almost saturated at the concentrations shown by the invention (inventive compositions 1-2-3, -4).

Similar concentration dependency was found also on respective amino acids (inventive compositions 1-2-5 to 1-2-34). Also, growth of human epidermal keratinocyte was suppressed by the medium to which the amino acids of the invention were not added at all (comparative compositions 1-2-1 to 1-2-15).

In addition, the growth acceleration effect was not found by conventionally known combinations of amino acids (comparative compositions 1-2-16 and 1-2-17).

### (Cosmetics Test)

### Reference Example 1-1

A skin care lotion was produced by the following composition as an experiment.

Respective components were made into aqueous solution by mixing them at 70° C. to the concentrations shown in the following Table 1-28, and then cooled to room temperature.

TABLE 1-28
Skin care lotion

| | Inventive composition |
|---|---|
| Components | Concentration (μg/ml) |
| Arginine | 80 |
| Aspartic acid | 15 |
| N-acetylglutamine | 100 |
| Glycine | 30 |
| Histidine | 40 |
| Isoleucine | 100 |
| Leucine | 100 |
| Lysine | 100 |
| Hydroxyproline | 30 |
| Serine | 40 |
| Threonine | 90 |
| Valine | 90 |
| Tyrosine | 0.4 |
| Cysteine | 50 |
| Phenylalanine | 0.6 |
| Vitamin B₆ | 4 |
| Vitamin B₁ | 4 |
| Hydrolyzed silk | 50 |
| Citric acid | 30 |
| Na hyaluronate | 500 |
| Xylitol | 1000 |
| 1,3-BG | 50000 |
| water | up to |

### Reference Example 1-2

An emulsion for skin care was produced by the following composition as an experiment.

### Solution A

Respective components were made into aqueous solution by mixing them at 70° C. to the concentrations shown in Table 1-29.

TABLE 1-29
Solution A

| | Inventive composition |
|---|---|
| Components | Concentration (μg/ml) |
| Arginine | 160 |
| Aspartic acid | 30 |
| N-acetylglutamine | 200 |
| Glycine | 60 |
| Histidine | 80 |
| Isoleucine | 200 |
| Leucine | 200 |
| Lysine | 200 |
| Hydroxyproline | 60 |
| Serine | 80 |
| Threonine | 180 |
| Valine | 180 |
| Tyrosine | 0.8 |
| Cysteine | 100 |
| Phenylalanine | 1.2 |
| Vitamin B₆ | 8 |
| Vitamin B₁ | 8 |
| Hydrolyzed silk | 100 |
| Citric acid | 60 |
| Na hyaluronate | 1000 |
| Xylitol | 2000 |
| Ethanol | 10000 |
| Lipidure-PMB | 4000 |
| (mfd. by Nippon Oil & Fats) | |
| water | up to |

### Solution B

Respective components were mixed at 70° C. to the composition ratio shown in Table 1-30.

TABLE 1-30
Solution B

| Components | Weight ratio |
|---|---|
| Squalane | 50 |
| 1,3-BG | 50 |
| Petrolatum | 20 |
| Polyoxyethylene oleyl maleyl | 12 |
| Sorbitan sesquioleic acid ester | 8 |
| Beeswax | 5 |

### Production Method

A 65 ml portion of the solution A and 15 g of the solution B were mixed at 70° C., further mixed by adding 20 ml of xanthan gum (2% aqueous solution) until they became uniform, and then cooled to room temperature.

### Reference Example 1-3

A cream for skin care was produced by the following composition as an experiment.

### Solution A

Respective components were made into aqueous solution by mixing them at 70° C. to obtain the composition shown in Table 1-31.

TABLE 1-31
Solution A

| | Inventive composition |
|---|---|
| Components | Concentration (μg/ml) |
| Arginine | 160 |
| Aspartic acid | 30 |
| N-acetylglutamine | 200 |
| Glycine | 60 |
| Histidine | 80 |
| Isoleucine | 200 |
| Leucine | 200 |
| Lysine | 200 |
| Hydroxyproline | 60 |
| Serine | 80 |
| Threonine | 180 |
| Valine | 180 |
| Tyrosine | 0.8 |
| Cysteine | 100 |
| Phenylalanine | 1.2 |
| Vitamin B₆ | 8 |
| Vitamin B₁ | 8 |
| Hydrolyzed silk | 100 |
| Citric acid | 60 |
| Na hyaluronate | 1000 |
| Xylitol | 2000 |
| Ethanol | 10000 |
| Lipidure-PMB | 4000 |
| (mfd. by Nippon Oil & Fats) | |
| water | up to |

### Solution B

Respective components were mixed at 70° C. to the composition ratio shown in Table 1-32.

TABLE 1-32
Solution B

| Components | Weight ratio |
|---|---|
| Cetyl alcohol | 5 |
| Stearic acid | 3 |
| Petrolatum | 5 |
| Squalane | 5 |
| Trioctanoin | 10 |
| Propylene glycol stearate | 7 |
| CETETH-20 (mfd. by Nihon Emulsion) | 3 |

### Production Method

A 51 ml portion of the solution A and 40 g of the solution B were mixed at 70° C., further mixed by adding 1.0 g of triethanolamine at 70° C. until they were uniformly emulsified, and then cooled to room temperature.

### Inventive Example 1-3

Skin care lotion
Evaluation method
Panel undergoing the test

Ten healthy women of from 27 to 35 years, 31.6 years in average.

### Place

The interior of a room controlled at a temperature of about 24° C. and a humidity of about 55%.

### Evaluation Method

Each sample was applied to a random position on the hidden side of a forearm after washing, and the used feeling (sensory feeling) was scored as a result of the sensory test based on the following criteria.

4: Feels very good

3. Feels moderately good

2: Feels not so good

1: No feeling

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

### Skin Care Lotion

The composition of Table 1-28 was regarded as this invention, the same composition from which acetylglutamine was removed was regarded as Comparative Example 1-3-1, and from which hydroxyproline was removed as Comparative Example 1-3-2.

TABLE 1-33
Skin care lotion

| | | Comparative | Comparative |
|---|---|---|---|
| Added component | This invention | Example 1-3-1 | Example 1-3-2 |
| Acetylglutamine | + | − | + |
| Hydroxyproline | + | + | − |

About 5 minutes after the application by sufficient rubbing, their impressions were asked, with the results shown in the following.

TABLE 1-34
Results of skin care lotion sensory test

| | Penetrated | | | Overall |
|---|---|---|---|---|
| | feel | Moist feel | Spread feel | evaluation |
| This invention | A | A | A | A |
| Comparative | C | C | B | B |
| Example 1-3-1 | | | | |
| Comparative | C | C | B | C |
| Example 1-3-2 | | | | |

As is evident from the table, the lotion of this invention in which specified amino acids were used jointly with acetylglutamine and hydroxyproline is a cosmetic which shows strong penetrated feel and superior moist feel and spread feel and is also excellent synthetically.

### Inventive Example 1-4

### Moisture Keeping Effect

The evaluation panel was as described in the above, and their average age was 33.8 years.

The moisture keeping effect was evaluated by measuring the horny layer moisture by a high frequency impedance method. (A double frequency phase difference amplitude detection system using a high sensitivity horny layer film thickness moisture meter, ASA-MX, mfd. by Asahi Bio Med)

### Skin Care Emulsion

The composition of Table 1-29 was regarded as this invention, the same composition from which acetylglutamine was removed was regarded as Comparative Example 1-4-1, and from which hydroxyproline was removed as Comparative Example 1-4-2.

TABLE 1-35
Skin care emulsion

| | This | Comparative | Comparative |
|---|---|---|---|
| Added component | invention | Example 1-4-1 | Example 1-4-2 |
| Acetylglutamine | + | − | + |
| Hydroxyproline | + | + | − |

About 18 hours after the application by sufficient rubbing, the skin conductivity was measured to calculate its increasing ratio after the application.

TABLE 1-36
Skin care emulsion

| | Increasing ratio |
|---|---|
| This invention | 59 |
| Comparative Example 1-4-1 | 42 |
| Comparative Example 1-4-2 | 40 |

As is evident from the table, the composition of the invention showed excellent moisture keeping property.

### Inventive Example 1-5

### Evaluation Method

The evaluation panel was as described in the above, and their average age was 32.1 years.

After washing both hands in the morning and noon every day, each sample was applied to the back and used continuously for 2 weeks to carry out a used effect test.

The test results were scored based on the following criteria.

### Skin Activation Effect

4: Improvement of tension and gloss of the skin is felt strongly

3. Felt moderately

2: Felt slightly

1: Not felt

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

### Chapped Skin Suppressing Effect

4: Improvement of dryness of the skin and chapped skin is felt strongly

3. Felt moderately

2: Felt slightly

1: Not felt

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

The composition of Table 1-31 was regarded as this invention, the same composition from which acetylglutamine was removed was regarded as Comparative Example 1-5-1, and from which hydroxyproline was removed as Comparative Example 1-5-2.

TABLE 1-37
Skin care cream

| | This | Comparative | Comparative |
|---|---|---|---|
| Added component | invention | Example 1-5-1 | Example 1-5-2 |
| Acetylglutamine | + | − | + |
| Hydroxyproline | + | + | − |

TABLE 1-38
Skin care cream

| | Skin | Chapped skin |
|---|---|---|
| | activation | suppression |
| This invention | A | A |
| Comparative Example 1-5-1 | B | C |
| Comparative Example 1-5-2 | B | C |

As is evident from Table 1-38, the composition of the invention showed excellent skin activating and chapped skin suppressing effects.

### Inventive Example 2-1

### Skin Permeation Test

### The Skin for Permeation Test

The abdominal skin of each of male hairless mice of 10 to 20 weeks of age (purchased from Kyudo) was extracted and the subcutaneous fat was removed therefrom, and this was used as the skin.

### Skin Permeation Test

The skin sample was attached to a Keshany-Chien type diffusion cell. A 1 ml portion of each test liquid (described later) was added to the donor phase, and its upper side was sealed with Para Film. A 0.01 mol/l phosphate buffered saline (Wako Pure Chemical Industries) was added to the receptor phase, stirred and incubated at 32° C., and amounts of amino acids were determined by an HPLC method 24 hours later to calculate permeation ratio.

### Amino Acid Determination Method

Using an HPLC amino acid analysis system of Shimadzu Corp. (Prominence), the determination was carried out by a post column fluorescence detection method which uses OPA (ortho-phthalaldehyde) as a reaction reagent.

### Test Liquids

A test liquid I was prepared by mixing 20 amino acid species (valine, leucine, isoleucine, alanine, arginine, glutamine, lysine, aspartic acid, glutamic acid, proline, cysteine, threonine, methionine, histidine, phenylalanine, tyrosine, tryptophan, asparagine, glycine and serine; mfd. by Wako Pure Chemical Industries) respectively to a final concentration of 0.1% by weight, 1,3-butylene glycol (Wako Pure Chemical Industries) to a final concentration of 1% by weight and a hydrolyzed silk (Promoi Silk: mfd. by Seiwa Kasei) as an additive agent to a final concentration of 1% by weight, with 0.01 mol/l phosphate buffered saline (Wako Pure Chemical Industries).

Test liquids 2, 3, 4, 5 and 6 were prepared by respectively mixing glucose (Wako Pure Chemical Industries), trehalose (Wako Pure Chemical Industries), a marine collagen (Marigen S-06, mfd. by Nitta Gelatin), a marine collagen (Marigen SP-03, Nitta Gelatin) or a swine dermis-derived collagen (Collagen P, Nitta Gelatin) in the same manner, instead of the hydrolyzed silk.

The test liquids were tested by the aforementioned skin permeation test method, and permeability of the permeated whole amino acids was measured, with the results shown in Table 2-1.

TABLE 2-1
Skin permeation test

| Additive component | Permeability (%) | |
|---|---|---|
| Hydrolyzed silk | 14.0 | Test liquid 1 (Inventive) |
| Glucose | 9.6 | Test liquid 2 (Comparative) |
| Trehalose | 7.2 | Test liquid 3 (Comparative) |
| Marigen S-06 | 9.1 | Test liquid 4 (Comparative) |
| Marigen SP-03 | 8.9 | Test liquid 5 (Comparative) |
| Collagen P | 8.3 | Test liquid 6 (Comparative) |

The hydrolyzed silk of the invention showed its permeation accelerating effect superior to those of the saccharides glucose and trehalose, and also showed its permeation accelerating effect superior to those of other hydrolyzed peptides.

Permeability of each amino acid when the additive agent of the invention is hydrolyzed silk is shown in Table 2-2.

TABLE 2-2

| | |
|---|---|
| Valine | 14.9 |
| Leucine | 21.3 |
| Isoleucine | 20.8 |
| Alanine | 8.4 |
| Arginine | 18.1 |
| Glutamine | 10.2 |
| Lysine | 19.6 |
| Aspartic acid | 19.1 |
| Glutamic acid | 5.5 |
| Proline | 7.9 |
| Cysteine | 14.7 |
| Threonine | 19.3 |
| Methionine | 7.4 |
| Histidine | 15.2 |
| Phenylalanine | 15.1 |
| Tyrosine | 15.3 |
| Tryptophan | 3.6 |
| Asparagine | 14.2 |
| Glycine | 14.3 |
| Serine | 15.2 |

Leucine, isoleucine, arginine, lysine, aspartic acid and threonine showed superior skin permeability under the test conditions in the presence of hydrolyzed silk, and this suggests a possibility that permeation into the cells is effected by the synergistic effect of these amino acids with hydrolyzed silk.

### Inventive Example 2-2

### Human Normal Keratinocyte Growth Test

### Preparation of Purified IV Type Collagen-Acetic Acid Solution

In accordance with the following protocols (1) to (16), 8 mg of a purified high polymer IV type collagen was obtained from a swine eyeball as the material. The following protocols were carried out at 4° C.
(1) The cornea is removed from the eyeball using scissors, and the lens is fished out from inside of the eyeball.
(2) Vitreous body and the like insoluble parts adhered to the lens are removed as many as possible using scissors and the like.
(3) One tablet of Complete Protease Inhibitor Cocktail (mfd. by Roche) is added to 50 ml of cold PBS (phosphate buffered saline) and dissolved therein, and the lens capsule is put therein and stirred for 2 hours.
(4) By carrying out centrifugation (2,000 g, 10 minutes, 4° C.), the unnecessary parts presenting in the supernatant are removed.
(5) The precipitate is suspended in 25 ml of 0.5 M acetic acid in which half a tablet of Complete Protease Inhibitor Cocktail (Roche) was dissolved in advance.
(6) This is finely crashed using a homogenizer (IKA).
(7) The finely crashed lens capsule is stirred for 3 days, and extraction of IV type collagen is carried out.
(8) By carrying out centrifugation (2,000 g, 10 minutes, 4° C.), the supernatant (acetic acid-soluble collagen) and the precipitate are separated.
(9) This extraction by stirring and the centrifugation are repeated again.
(10) Crystals of NaCI are ground down as fine as possible using a mortar and added to the supernatant obtained by centrifugation to a final concentration of 1.7 M.
(11) This is stirred overnight to effect precipitation of collagen.
(12) This is centrifuged (5,000 g, 30 minutes, 4° C.), and the precipitate is recovered.
(13) The precipitate is mixed with 0.5 M acetic acid and sufficiently dissolved therein.
(14) The acidic aqueous solution of collagen is put into a dialysis tube (Sanko Junyaku), and its dialysis is carried out using 0.5 M acetic acid.
(15) The dialysis is carried out again using 2 mM hydrochloric acid.
(16) The collagen solution after dialysis is recovered to obtain a solution of purified IV type collagen.

### Operating Procedure of Culturing on a Collagen Film

1. The aforementioned IV type collagen-acetic acid solution (1 mg/ml) is diluted 10 times with sterilized ultrapure water and poured into a 48 well culture dish or plate to a portion of from 50 to 100 μl per 1 cm².
2. This is thinly stretched such that it is spread out over the whole culture face.
3. This is dried at 25° C. or less, by opening the cover of the culture dish or plate in a clean bench.
4. After the drying, this is washed 3 times with the medium in order to remove the acid from the IV type collagen film.
5. Human normal keratinocyte (CC-2503-NZ, Sanko Junyaku) is inoculated to a density of 3,500 cells per 1 cm².
6. The IV collagen plate is put into a CO₂ incubator to start the culturing.
7. This is cultured for 8 days by changing the medium at an interval of 2 days.

### Skin Cell Growth Test

The growth test was carried out using MTT Cell Growth Assay Kit.

Reagent A: MTT, (3-(4,5-dimethylthiazol-2-yl)-2,5-diphenyl tetrasodium bromide), 50 mg/vial

Reagent B: PBS pH 7.4, 15 ml

Reagent C: color development solution (isopropanol with 0.04 N HCl), 100 ml

A 10 ml portion of the reagent B is added to the reagent A and thoroughly mixed.

This is allowed to stand overnight in a cool and dark space.

The AB reagent is sterilized by filtration through a 0.22 μm filter.

The AB reagent is added in a portion of 30 μl per 1 cm² and lightly mixed.

This is cultured for 4 hours in a CO₂ incubator.

After the culturing, the culture supernatant is transferred into a tube.

The reagent C is added in a portion of 300 μl per 1 cm².

After mixing, this is transferred into the culture supernatant-containing tube.

The absorbance (570 nm) is measured within 1 hour.

### Test Medium

Respective additives (Wako Pure Chemical Industries) were added to a Bredt Kit KGM-2 medium (a growth medium for epidermal keratinocyte) Sanko Junyaku CC-1307-NZ, to the concentrations shown in Tables 2-3 to 2-15, and used as the test medium. In this connection, the test medium of Table 2-13 is a medium similar to the conventionally known composition shown in the examples of JP-A-61-289016.

TABLE 2-3
Test medium

| | Inventive | Inventive | Inventive | Inventive |
|---|---|---|---|---|
| | composition | composition | composition | composition |
| | 2-2-1 | 2-2-2 | 2-2-3 | 2-2-4 |
| | Con- | Con- | Con- | Con- |
| | centration | centration | centration | centration |
| Components | (μg/ml) | (μg/ml) | (μg/ml) | (μg/ml) |
| Arginine | 80 | 80 | 80 | 20 |
| Aspartic acid | 15 | 15 | 15 | 15 |
| Isoleucine | 100 | 100 | 100 | 100 |
| Leucine | 100 | 100 | 100 | 100 |
| Lysine | 100 | 100 | 100 | 100 |
| Threonine | 90 | 90 | 90 | 90 |
| Hydrolyzed | 50 | 50 | 50 | 50 |
| silk | | | | |
| Vitamin B₆ | 0 | 4 | 4 | 4 |
| Vitamin B₁ | 0 | 4 | 4 | 4 |
| Glycine | 0 | 0 | 30 | 30 |
| Histidine | 0 | 0 | 40 | 40 |
| Serine | 0 | 0 | 40 | 40 |
| Valine | 0 | 0 | 90 | 90 |
| Tyrosine | 0 | 0 | 0.4 | 0.4 |
| Cysteine | 0 | 0 | 50 | 50 |
| Phenylalanine | 0 | 0 | 0.6 | 0.6 |

TABLE 2-4
Test medium

| | Inventive | Inventive | Inventive | Inventive |
|---|---|---|---|---|
| | composition | composition | composition | composition |
| | 2-2-5 | 2-2-6 | 2-2-7 | 2-2-8 |
| | Con- | Con- | Con- | Con- |
| | centration | centration | centration | centration |
| Components | (μg/ml) | (μg/ml) | (μg/ml) | (μg/ml) |
| Arginine | 200 | 80 | 80 | 80 |
| Aspartic acid | 15 | 6 | 60 | 15 |
| Isoleucine | 100 | 50 | 600 | 100 |
| Leucine | 100 | 100 | 100 | 30 |
| Lysine | 100 | 100 | 100 | 100 |
| Threonine | 90 | 90 | 90 | 90 |
| Hydrolyzed | 50 | 50 | 50 | 50 |
| silk | | | | |
| Vitamin B₆ | 4 | 4 | 4 | 4 |
| Vitamin B₁ | 4 | 4 | 4 | 4 |
| Glycine | 30 | 30 | 30 | 30 |
| Histidine | 40 | 40 | 40 | 40 |
| Serine | 40 | 40 | 40 | 40 |
| Valine | 90 | 90 | 90 | 90 |
| Tyrosine | 0.4 | 0.4 | 0.4 | 0.4 |
| Cysteine | 50 | 50 | 50 | 50 |
| Phenylalanine | 0.6 | 0.6 | 0.6 | 0.6 |

TABLE 2-5
Test medium

| | Inventive | Inventive | Inventive | Inventive |
|---|---|---|---|---|
| | composition | composition | composition | composition |
| | 2-2-9 | 2-2-10 | 2-2-11 | 2-2-12 |
| | Con- | Con- | Con- | Con- |
| | centration | centration | centration | centration |
| Components | (μg/ml) | (μg/ml) | (μg/ml) | (μg/ml) |
| Arginine | 80 | 80 | 80 | 80 |
| Aspartic acid | 15 | 15 | 15 | 15 |
| Isoleucine | 100 | 100 | 100 | 100 |
| Leucine | 600 | 100 | 100 | 100 |
| Lysine | 100 | 30 | 600 | 100 |
| Threonine | 90 | 90 | 90 | 30 |
| Hydrolyzed | 50 | 50 | 50 | 50 |
| silk | | | | |
| Vitamin B₆ | 4 | 4 | 4 | 4 |
| Vitamin B₁ | 4 | 4 | 4 | 4 |
| Glycine | 30 | 30 | 30 | 30 |
| Histidine | 40 | 40 | 40 | 40 |
| Serine | 40 | 40 | 40 | 40 |
| Valine | 90 | 90 | 90 | 90 |
| Tyrosine | 0.4 | 0.4 | 0.4 | 0.4 |
| Cysteine | 50 | 50 | 50 | 50 |
| Phenylalanine | 0.6 | 0.6 | 0.6 | 0.6 |

TABLE 2-6
Test medium

| | Inventive | Inventive | Inventive | Inventive |
|---|---|---|---|---|
| | composition | composition | composition | composition |
| | 2-2-13 | 2-2-14 | 2-2-15 | 2-2-16 |
| | Con- | Con- | Con- | Con- |
| | centration | centration | centration | centration |
| Components | (μg/ml) | (μg/ml) | (μg/ml) | (μg/ml) |
| Arginine | 80 | 80 | 120 | 60 |
| Aspartic acid | 15 | 15 | 20 | 15 |
| Isoleucine | 100 | 100 | 150 | 80 |
| Leucine | 100 | 100 | 150 | 80 |
| Lysine | 100 | 100 | 150 | 80 |
| Threonine | 200 | 90 | 150 | 90 |
| Hydrolyzed | 50 | 5 | 200 | 200 |
| silk | | | | |
| Vitamin B₆ | 4 | 0 | 0 | 0 |
| Vitamin B₁ | 4 | 0 | 0 | 0 |
| Glycine | 30 | 0 | 0 | 0 |
| Histidine | 40 | 0 | 0 | 0 |
| Serine | 40 | 0 | 0 | 0 |
| Valine | 90 | 0 | 0 | 0 |
| Tyrosine | 0.4 | 0 | 0 | 0 |
| Cysteine | 50 | 0 | 0 | 0 |
| Phenylalanine | 0.6 | 0 | 0 | 0 |

TABLE 2-7
Test medium

| | Inventive | Inventive | Inventive | Inventive |
|---|---|---|---|---|
| | composition | composition | composition | composition |
| | 2-2-17 | 2-2-18 | 2-2-19 | 2-2-20 |
| | Con- | Con- | Con- | Con- |
| | centration | centration | centration | centration |
| Components | (μg/ml) | (μg/ml) | (μg/ml) | (μg/ml) |
| Arginine | 80 | 50 | 80 | 120 |
| Aspartic acid | 20 | 8 | 8 | 50 |
| Isoleucine | 150 | 60 | 50 | 100 |
| Leucine | 150 | 60 | 60 | 100 |
| Lysine | 120 | 60 | 60 | 80 |
| Threonine | 90 | 90 | 120 | 110 |
| Hydrolyzed | 200 | 1000 | 200 | 500 |
| silk | | | | |
| Vitamin B₆ | 6 | 6 | 4 | 4 |
| Vitamin B₁ | 4 | 6 | 4 | 4 |
| Glycine | 0 | 0 | 30 | 20 |
| Histidine | 0 | 0 | 40 | 50 |
| Serine | 0 | 0 | 40 | 60 |
| Valine | 0 | 0 | 90 | 70 |
| Tyrosine | 0 | 0 | 0.4 | 0.3 |
| Cysteine | 0 | 0 | 50 | 40 |
| Phenylalanine | 0 | 0 | 0.6 | 0.8 |

TABLE 2-8
Test medium

| | Inventive | Inventive | Inventive | Inventive |
|---|---|---|---|---|
| | composition | composition | composition | composition |
| | 2-2-21 | 2-2-22 | 2-2-23 | 2-2-24 |
| | Con- | Con- | Con- | Con- |
| | centration | centration | centration | centration |
| Components | (μg/ml) | (μg/ml) | (μg/ml) | (μg/ml) |
| Arginine | 30 | 20 | 40 | 50 |
| Aspartic acid | 20 | 5 | 40 | 20 |
| Isoleucine | 200 | 30 | 120 | 200 |
| Leucine | 200 | 30 | 150 | 120 |
| Lysine | 200 | 40 | 150 | 120 |
| Threonine | 150 | 30 | 80 | 50 |
| Hydrolyzed | 2000 | 2000 | 1000 | 10 |
| silk | | | | |
| Vitamin B₆ | 4 | 4 | 4 | 4 |
| Vitamin B₁ | 4 | 4 | 4 | 4 |
| Glycine | 30 | 30 | 30 | 30 |
| Histidine | 40 | 40 | 40 | 40 |
| Serine | 40 | 40 | 40 | 40 |
| Valine | 90 | 90 | 90 | 90 |
| Tyrosine | 0.4 | 0.4 | 0.4 | 0.4 |
| Cysteine | 50 | 50 | 50 | 50 |
| Phenylalanine | 0.6 | 0.6 | 0.6 | 0.6 |

TABLE 2-9
Test medium

| | Inventive | Inventive | Inventive | Inventive |
|---|---|---|---|---|
| | composition | composition | composition | composition |
| | 2-2-25 | 2-2-26 | 2-2-27 | 2-2-28 |
| | Con- | Con- | Con- | Con- |
| | centration | centration | centration | centration |
| Components | (μg/ml) | (μg/ml) | (μg/ml) | (μg/ml) |
| Arginine | 40 | 300 | 120 | 60 |
| Aspartic acid | 30 | 8 | 20 | 12 |
| Isoleucine | 50 | 400 | 200 | 80 |
| Leucine | 200 | 400 | 200 | 80 |
| Lysine | 200 | 80 | 200 | 80 |
| Threonine | 150 | 60 | 120 | 60 |
| Hydrolyzed | 20 | 20 | 200 | 20 |
| silk | | | | |
| Vitamin B₆ | 4 | 4 | 4 | 4 |
| Vitamin B₁ | 4 | 4 | 4 | 4 |
| Glycine | 30 | 30 | 30 | 30 |
| Histidine | 40 | 40 | 40 | 40 |
| Serine | 40 | 40 | 40 | 40 |
| Valine | 90 | 90 | 90 | 90 |
| Tyrosine | 0.4 | 0.4 | 0.4 | 0.4 |
| Cysteine | 50 | 50 | 50 | 50 |
| Phenylalanine | 0.6 | 0.6 | 0.6 | 0.6 |

TABLE 2-10
Test medium

| | Comparative | Comparative |
|---|---|---|
| | composition | composition |
| | 2-2-1 | 2-2-2 |
| | Concentration | Concentration |
| Components | (μg/ml) | (μg/ml) |
| Arginine | 80 | 80 |
| Aspartic acid | 15 | 15 |
| Isoleucine | 100 | 100 |
| Leucine | 100 | 100 |
| Lysine | 100 | 100 |
| Threonine | 90 | 90 |
| Hydrolyzed silk | 3 | 3 |
| Vitamin B₆ | 0 | 4 |
| Vitamin B₁ | 0 | 4 |
| Glycine | 0 | 30 |
| Histidine | 0 | 40 |
| Serine | 0 | 40 |
| Valine | 0 | 90 |
| Tyrosine | 0 | 0.4 |
| Cysteine | 0 | 50 |
| Phenylalanine | 0 | 0.6 |

TABLE 2-11
Test medium

| | Comparative | Comparative | Comparative | Comparative |
|---|---|---|---|---|
| | composition | composition | composition | composition |
| | 2-2-3 | 2-2-4 | 2-2-5 | 2-2-6 |
| | Con- | Con- | Con- | Con- |
| | centration | centration | centration | centration |
| Components | (μg/ml) | (μg/ml) | (μg/ml) | (μg/ml) |
| Arginine | 0 | 80 | 80 | 80 |
| Aspartic acid | 15 | 0 | 15 | 15 |
| Isoleucine | 100 | 100 | 0 | 100 |
| Leucine | 100 | 100 | 100 | 0 |
| Lysine | 100 | 100 | 100 | 100 |
| Threonine | 90 | 90 | 90 | 90 |
| Hydrolyzed | 200 | 200 | 200 | 200 |
| silk | | | | |
| Vitamin B₆ | 4 | 4 | 4 | 4 |
| Vitamin B₁ | 4 | 4 | 4 | 4 |
| Glycine | 30 | 30 | 30 | 30 |
| Histidine | 40 | 40 | 40 | 40 |
| Serine | 40 | 40 | 40 | 40 |
| Valine | 90 | 90 | 90 | 90 |
| Tyrosine | 0.4 | 0.4 | 0.4 | 0.4 |
| Cysteine | 50 | 50 | 50 | 50 |
| Phenylalanine | 0.6 | 0.6 | 0.6 | 0.6 |

TABLE 2-12
Test medium

| | Comparative | Comparative |
|---|---|---|
| | composition | composition |
| | 2-2-7 | 2-2-8 |
| | Concentration | Concentration |
| Components | (μg/ml) | (μg/ml) |
| Arginine | 80 | 80 |
| Aspartic acid | 15 | 15 |
| Isoleucine | 100 | 100 |
| Leucine | 100 | 100 |
| Lysine | 0 | 100 |
| Threonine | 90 | 0 |
| Hydrolyzed silk | 200 | 200 |
| Vitamin B₆ | 4 | 4 |
| Vitamin B₁ | 4 | 4 |
| Glycine | 30 | 30 |
| Histidine | 40 | 40 |
| Serine | 40 | 40 |
| Valine | 90 | 90 |
| Tyrosine | 0.4 | 0.4 |
| Cysteine | 50 | 50 |
| Phenylalanine | 0.6 | 0.6 |

TABLE 2-13
Test medium

| | Comparative | Comparative | Comparative |
|---|---|---|---|
| | composition | composition | composition |
| | 2-2-9 | 2-2-10 | 2-2-11 |
| | Concentration | Concentration | Concentration |
| Components | (μg/ml) | (μg/ml) | (μg/ml) |
| Isoleucine | 50 | 25 | 0 |
| Tryptophan | 25 | 12.5 | 0 |
| Threonine | 50 | 25 | 0 |
| Valine | 50 | 25 | 100 |
| Phenylalanine | 25 | 12.5 | 0 |
| Methionine | 25 | 12.5 | 0 |
| Lysine | 75 | 37.5 | 100 |
| Leucine | 50 | 25 | 0 |
| Glutamine | 300 | 150 | 100 |
| Myo-inositol | 3.5 | 1.75 | 2 |
| Vitamin B₆ | 2 | 1 | 0 |
| Pantothenic acid | 2 | 1 | 0 |
| Nicotinamide | 2 | 1 | 1 |
| Glucose | 500 | 250 | 1000 |
| Succinic acid | 0.5 | 0.25 | 0 |

TABLE 2-14
Test medium

| | Comparative | Comparative | Comparative |
|---|---|---|---|
| | composition | composition | composition |
| | 2-2-12 | 2-2-13 | 2-2-14 |
| | Concentration | Concentration | Concentration |
| Components | (μg/ml) | (μg/ml) | (μg/ml) |
| Arginine | 10 | 15 | 60 |
| Aspartic acid | 30 | 20 | 15 |
| Isoleucine | 10 | 150 | 10 |
| Leucine | 200 | 10 | 10 |
| Lysine | 200 | 150 | 10 |
| Threonine | 90 | 90 | 90 |
| Hydrolyzed silk | 200 | 200 | 100 |
| Vitamin B₆ | 4 | 4 | 4 |
| Vitamin B₁ | 4 | 4 | 4 |
| Glycine | 30 | 30 | 30 |
| Histidine | 40 | 40 | 40 |
| Serine | 40 | 40 | 40 |
| Valine | 90 | 90 | 90 |
| Tyrosine | 0.4 | 0.4 | 0.4 |
| Cysteine | 50 | 50 | 50 |
| Phenylalanine | 0.6 | 0.6 | 0.6 |

TABLE 2-15
Test medium

| | Comparative | Comparative | Comparative |
|---|---|---|---|
| | composition | composition | composition |
| | 2-2-15 | 2-2-16 | 2-2-17 |
| | Concentration | Concentration | Concentration |
| Components | (μg/ml) | (μg/ml) | (μg/ml) |
| Arginine | 80 | 80 | 10 |
| Aspartic acid | 3 | 3 | 2 |
| Isoleucine | 10 | 10 | 50 |
| Leucine | 100 | 200 | 10 |
| Lysine | 100 | 150 | 20 |
| Threonine | 10 | 90 | 20 |
| Hydrolyzed silk | 80 | 5 | 5 |
| Vitamin B₆ | 4 | 4 | 4 |
| Vitamin B₁ | 4 | 4 | 4 |
| Glycine | 30 | 30 | 30 |
| Histidine | 40 | 40 | 40 |
| Serine | 40 | 40 | 40 |
| Valine | 90 | 90 | 90 |
| Tyrosine | 0.4 | 0.4 | 0.4 |
| Cysteine | 50 | 50 | 50 |
| Phenylalanine | 0.6 | 0.6 | 0.6 |

### Results of Skin Cell Growth Test

By regarding the absorbance at the time of no addition as 100, corresponding relative concentrations were calculated, with the results shown in Tables 2-16 and 2-17.

TABLE 2-16
Human epidermal keratinocyte growth accelerating effect

| Medium | Relative concentration |
|---|---|
| No addition | 100 |
| Inventive composition 2-2-1 | 108 |
| Inventive composition 2-2-2 | 119 |
| Inventive composition 2-2-3 | 125 |
| Inventive composition 2-2-4 | 106 |
| Inventive composition 2-2-5 | 127 |
| Inventive composition 2-2-6 | 107 |
| Inventive composition 2-2-7 | 126 |
| Inventive composition 2-2-8 | 110 |
| Inventive composition 2-2-9 | 129 |
| Inventive composition 2-2-10 | 107 |
| Inventive composition 2-2-11 | 121 |
| Inventive composition 2-2-12 | 110 |
| Inventive composition 2-2-13 | 121 |
| Inventive composition 2-2-14 | 107 |
| Inventive composition 2-2-15 | 110 |
| Inventive composition 2-2-16 | 108 |
| Inventive composition 2-2-17 | 117 |
| Inventive composition 2-2-18 | 116 |
| Inventive composition 2-2-19 | 121 |
| Inventive composition 2-2-20 | 122 |
| Inventive composition 2-2-21 | 110 |
| Inventive composition 2-2-22 | 107 |
| Inventive composition 2-2-23 | 115 |
| Inventive composition 2-2-24 | 108 |
| Inventive composition 2-2-25 | 110 |
| Inventive composition 2-2-26 | 111 |
| Inventive composition 2-2-27 | 125 |
| Inventive composition 2-2-28 | 122 |

TABLE 2-17
Human epidermal keratinocyte growth accelerating effect

| Medium | Relative concentration |
|---|---|
| No addition | 100 |
| Comparative composition 2-2-1 | 101 |
| Comparative composition 2-2-2 | 103 |
| Comparative composition 2-2-3 | 86 |
| Comparative composition 2-2-4 | 82 |
| Comparative composition 2-2-5 | 87 |
| Comparative composition 2-2-6 | 82 |
| Comparative composition 2-2-7 | 83 |
| Comparative composition 2-2-8 | 85 |
| Comparative composition 2-2-9 | 103 |
| Comparative composition 2-2-10 | 102 |
| Comparative composition 2-2-11 | 101 |
| Comparative composition 2-2-12 | 101 |
| Comparative composition 2-2-13 | 99 |
| Comparative composition 2-2-14 | 100 |
| Comparative composition 2-2-15 | 98 |
| Comparative composition 2-2-16 | 102 |
| Comparative composition 2-2-17 | 99 |

The inventive composition 2-2-1 accelerated growth of human epidermal keratinocyte on the IV type collagen plate, and the effect was improved by vitamin B₆ and vitamin B₁ (inventive composition 2-2-2) and also improved by the addition of several amino acids (inventive composition 2-2-3).

Influences by the concentration of respective amino acids are shown in the inventive compositions 2-2-4 to 2-2-13. The cell growth effect was saturated substantially with the upper limit amount shown by each amino acid. Regarding the amount of hydrolyzed silk, there is a cell growth effect at 5 μg/ml (inventive composition 2-2-14), but the effect is lost when reduced to 3 μg/ml (comparative composition 2-2-1), and there is no cell growth effect when vitamin B species and amino acids are added (comparative composition 2-2-1).

When the amino acids of the invention were not added, the cell growth effect was not obtained at all (comparative compositions 2-2-3 to 2-2-8).

The human epidermal keratinocyte growth effect was not found by the conventionally known combination of amino acids alone (comparative compositions 2-2-9 to 2-2-11).

### (Cosmetics Test)

### Reference Example 2-1

A skin care lotion was produced by the following composition as an experiment.

Respective components were made into aqueous solution by mixing them at 70° C. to the concentrations shown in the following Table 2-18, and then cooled to room temperature.

TABLE 2-18
Skin care lotion

| | Inventive composition |
|---|---|
| Components | Concentration (μg/ml) |
| Arginine | 80 |
| Aspartic acid | 15 |
| Isoleucine | 100 |
| Leucine | 100 |
| Lysine | 100 |
| Threonine | 90 |
| Hydrolyzed silk | 50 |
| Vitamin B₆ | 4 |
| Vitamin B₁ | 4 |
| Glycine | 30 |
| Histidine | 40 |
| Serine | 40 |
| Valine | 90 |
| Tyrosine | 0.4 |
| Cysteine | 50 |
| Phenylalanine | 0.6 |
| Citric acid | 30 |
| Na hyaluronate | 500 |
| 1,3-BG | 50000 |

### Reference Example 2-2

An emulsion for skin care was produced by the following composition as an experiment.

### Solution A

Respective components were made into aqueous solution by mixing them at 70° C. to the concentrations shown in Table 2-19.

TABLE 2-19
Solution A

| | Inventive composition |
|---|---|
| Components | Concentration (μg/ml) |
| Arginine | 160 |
| Aspartic acid | 30 |
| Isoleucine | 200 |
| Leucine | 200 |
| Lysine | 200 |
| Threonine | 180 |
| Hydrolyzed silk | 100 |
| Vitamin B₆ | 8 |
| Vitamin B₁ | 8 |
| Glycine | 60 |
| Histidine | 80 |
| Serine | 80 |
| Valine | 180 |
| Tyrosine | 0.8 |
| Cysteine | 100 |
| Phenylalanine | 1.2 |
| Citric acid | 60 |
| Na hyaluronate | 1000 |
| Ethanol | 100000 |
| Lipidure-PMB | 4000 |
| (mfd. by Nippon Oil & Fats) | |

### Solution B

Respective components were mixed at 70° C. to the composition ratio shown in Table 2-20.

TABLE 2-20
Solution B

| Components | Weight ratio |
|---|---|
| Squalane | 50 |
| 1,3-BG | 50 |
| Petrolatum | 20 |
| Polyoxyethylene oleyl maleyl | 12 |
| Sorbitan sesquioleic acid ester | 8 |
| Beeswax | 5 |

### Production Method

A 65 ml portion of the solution A and 15 g of the solution B were mixed at 70° C., further mixed by adding 20 ml of xanthan gum (2% aqueous solution) at 70° C. until they became uniform, and then cooled to room temperature.

### Reference Example 2-3

A cream for skin care was produced by the following composition as an experiment.

### Solution A

Respective components were made into aqueous solution by mixing them at 70° C. to obtain the composition shown in Table 2-21.

TABLE 2-21
Solution A

| | Inventive composition |
|---|---|
| Components | Concentration (μg/ml) |
| Arginine | 160 |
| Aspartic acid | 30 |
| Isoleucine | 200 |
| Leucine | 200 |
| Lysine | 200 |
| Threonine | 180 |
| Hydrolyzed silk | 100 |
| Vitamin B₆ | 8 |
| Vitamin B₁ | 8 |
| Glycine | 60 |
| Histidine | 80 |
| Serine | 80 |
| Valine | 180 |
| Tyrosine | 0.8 |
| Cysteine | 100 |
| Phenylalanine | 1.2 |
| Citric acid | 60 |
| Na hyaluronate | 1000 |
| Glycerol | 100000 |
| Lipidure-PMB | 4000 |
| (mfd. by Nippon Oil & Fats) | |

### Solution B

Respective components were mixed at 70° C. to the composition ratio shown in Table 2-22.

TABLE 2-22
Solution B

| Components | Weight ratio |
|---|---|
| Cetyl alcohol | 5 |
| Stearic acid | 3 |
| Petrolatum | 5 |
| Squalane | 5 |
| Trioctanoin | 10 |
| Propylene glycol stearate | 7 |
| CETETH-20 (mfd. by Nihon Emulsion) | 3 |

### Production Method

A 51 ml portion of the solution A and 40 g of the solution B were mixed at 70° C., further mixed by adding 1.0 g of triethanolamine at 70° C. until they were uniformly emulsified, and then cooled to room temperature.

### Inventive Example 2-3

### Skin Care Lotion

### Evaluation Method

### Panel Undergoing the Test

Ten healthy women of from 27 to 35 years, 34.2 years in average.

### Place

The interior of a room controlled at a temperature of about 24° C. and a humidity of about 55%.

### Evaluation Method

Each sample was applied to a random position on the hidden side of a forearm after washing, and the used feeling (sensory feeling) was scored as a result of the sensory test based on the following criteria.

4: Feels very good

3. Feels moderately good

2: Feels not so good

1: No feeling

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

### Skin Care Lotion

The composition of Table 2-18 was regarded as this invention, the same composition from which hydrolyzed silk was removed was regarded as Comparative Example 2-3-1, in which hydrolyzed silk was replaced by a marine collagen (Maringen SP-03, mfd. by Nitta Gelatin) as Comparative Example 2-3-2, and in which it was replaced by a swine dermis-derived collagen (Collagen P, Nitta Gelatin) as Comparative Example 2-3-3.

TABLE 2-23
Skin care lotion

| | | Comparative | Comparative | Comparative |
|---|---|---|---|---|
| | This | Example | Example | Example |
| Added component | invention | 2-3-1 | 2-3-2 | 2-3-3 |
| Hydrolyzed silk | + | − | − | − |
| Maringen SP-03 | − | − | + | − |
| Collagen P | − | − | − | + |

About 5 minutes after the application by sufficient rubbing, their impressions were asked, with the results shown in the following.

TABLE 2-24
Results of skin care lotion sensory test

| | Penetrated | | | Overall |
|---|---|---|---|---|
| | feel | Moist feel | Spread feel | evaluation |
| This invention | A | A | A | A |
| Comparative | C | C | B | C |
| Example 2-3-1 | | | | |
| Comparative | C | C | B | C |
| Example 2-3-2 | | | | |
| Comparative | C | C | B | C |
| Example 2-3-3 | | | | |

As is evident from the table, the amino acid lotion of this invention in which hydrolyzed silk was jointly used is a cosmetic which shows strong penetrated feel and superior moist feel and spread feel and is also excellent synthetically.

### Inventive Example 2-4

### Moisture Keeping Effect

The evaluation panel was as described in the above, and their average age was 32.7 years.

The moisture keeping effect was evaluated by measuring the horny layer moisture by a high frequency impedance method. (A double frequency phase difference amplitude detection system using a high sensitivity horny layer film thickness moisture meter, ASA-MX, mfd. by Asahi Bio Med)

### Skin Care Emulsion

The composition of Table 2-19 from which hydrolyzed silk was removed was regarded as Comparative Example 2-4-1, and the composition in which hydrolyzed silk was replaced by a marine collagen (Maringen SP-03, mfd. by Nitta Gelatin) as Comparative Example 2-4-2, and in which it was replaced by a swine dermis-derived collagen (Collagen P, Nitta Gelatin) as Comparative Example 2-4-3.

TABLE 2-25
Skin care emulsion

| | | Comparative | Comparative | Comparative |
|---|---|---|---|---|
| | This | Example | Example | Example |
| Added component | invention | 2-4-1 | 2-4-2 | 2-4-3 |
| Hydrolyzed silk | + | − | − | − |
| Maringen SP-03 | − | − | + | − |
| Collagen P | − | − | − | + |

About 18 hours after the application by sufficient rubbing, the skin conductivity was measured to calculate its increasing ratio after the application.

TABLE 2-26
Skin care emulsion

| | Increasing ratio |
|---|---|
| This invention | 58 |
| Comparative Example 2-4-1 | 33 |
| Comparative Example 2-4-2 | 46 |
| Comparative Example 2-4-3 | 44 |

As is evident from the table, the composition of the invention showed excellent moisture keeping property.

### Inventive Example 2-5

### Evaluation Method

The evaluation panel was as described in the above, and their average age was 36.4 years.

After washing both hands in the morning and noon every day, each sample was applied to the back and used continuously for 2 weeks to carry out a used effect test.

The test results were scored based on the following criteria.

### Skin Activation Effect

4: Improvement of tension and gloss of the skin is felt strongly

3. Felt moderately

2: Felt slightly

1: Not felt

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

### Chapped Skin Suppressing Effect

4: Improvement of dryness of the skin and chapped skin is felt strongly

3. Felt moderately

2: Felt slightly

1: Not felt

Thereafter, average of the evaluation points of 10 panel members was calculated and ranked as follows.

A: 3.2 or more

B: 2.7 or more and less than 3.2

C: 2.2 or more and less than 2.7

D: 1.7 or more and less than 2.2

E: less than 1.7

The composition of Table 2-21 was regarded as this invention, the same composition from which hydrolyzed silk was removed was regarded as Comparative Example 2-5-1, and the composition in which hydrolyzed silk was replaced by a marine collagen (Maringen SP-03, mfd. by Nitta Gelatin) as Comparative Example 2-5-2, and in which it was replaced by a swine dermis-derived collagen (Collagen P, Nitta Gelatin) as Comparative Example 2-5-3.

TABLE 2-27
Skin care cream

| | | Comparative | Comparative | Comparative |
|---|---|---|---|---|
| | This | Example | Example | Example |
| Added component | invention | 2-5-1 | 2-5-2 | 2-5-3 |
| Hydrolyzed silk | + | − | − | − |
| Maringen SP-03 | − | − | + | − |
| Collagen P | − | − | − | + |

TABLE 2-28
Skin care cream

| | | Chapped skin |
|---|---|---|
| | Skin activation | suppression |
| This invention | A | A |
| Comparative Example 2-5-1 | C | C |
| Comparative Example 2-5-2 | C | B |
| Comparative Example 2-5-3 | C | C |

As is evident from the table, the composition of the invention showed excellent skin activating and chapped skin suppressing effects.

According to the invention, an external preparation which has the moisture keeping action and cell activation action and shows a permeating feeling can be provided.

The entire disclosure of each and every foreign patent application from which the benefit of foreign priority has been claimed in the present application is incorporated herein by reference, as if fully set forth.

What is claimed is:

1. A skin external preparation, which comprises at least arginine, aspartic acid, isoleucine, leucine, lysine, threonine, glycine, histidine, serine, valine, tyrosine, cysteine, phenylalanine, hydroxyproline and acylglutamine among amino acids, or salts thereof.

2. The skin external preparation according to claim 1,
wherein, with respect to adding amounts of the amino acids or the salts thereof, an adding amount of arginine is from 20 to 400μ% by weight, an adding amount of aspartic acid is from 3 to 60μ% by weight, an adding amount of isoleucine is from 30 to 600μ% by weight, an adding amount of leucine is from 30 to 600μ% by weight, an adding amount of lysine is from 30 to 600μ% by weight, an adding amount of threonine is from 25 to 500μ% by weight, an adding amount of glycine is from 6 to 120μ% by weight, an adding amount of histidine is from 8 to 160μ% by weight, an adding amount of serine is from 8 to 160μ% by weight, an adding amount of valine is from 30 to 500μ% by weight, an adding amount of tyrosine is from 0.08 to 20μ% by weight, an adding amount of cysteine is from 15 to 300μ% by weight, an adding amount of phenylalanine is from 0.12 to 20μ% by weight and an adding amount of hydroxyproline is from 5 to 150μ% by weight.

3. The skin external preparation according to claim 1,
wherein the acylglutamine is acetylglutamine.

4. The skin external preparation according to claim 3,
wherein an adding amount of the acetylglutamine is from 10 to 800μ% by weight.

5. The skin external preparation according to claim 1, which further comprises a vitamin B.

6. The skin external preparation according to claim 5,
wherein the vitamin B is vitamin B₁ or vitamin B₆.

7. The skin external preparation according to claim 1, which further comprises hyaluronic acid or a salt thereof.

8. The skin external preparation according to claim 1, which is a cosmetic or a quasi drug.

9. The skin external preparation according to claim 8, which is a face lotion, an emulsion, a cream, a hair tonic or a pack.

10. A skin external preparation, which comprises:
at least arginine, aspartic acid, isoleucine, leucine, lysine and threonine among amino acids, or salts thereof; and
a hydrolyzed silk.

11. The skin external preparation according to claim 10,
wherein, with respect to adding amounts of the amino acids or the salts thereof, an adding amount of arginine is from 20 to 400μ% by weight, an adding amount of aspartic acid is from 3 to 60μ% by weight, an adding amount of isoleucine is from 30 to 600μ% by weight, an adding amount of leucine is from 30 to 600μ% by weight, an adding amount of lysine is from 30 to 600μ% by weight and an adding amount of threonine is from 25 to 500μ% by weight.

12. The skin external preparation according to claim 10,
wherein an adding amount of the hydrolyzed silk is from 5 to 3,000μ% by weight.

13. The skin external preparation according to claim 10, which further comprises at least one of glycine, histidine, serine, valine, tyrosine, cysteine, phenylalanine and salts thereof, among amino acids.

14. The skin external preparation according to claim 10, which further comprises a vitamin B.

15. The skin external preparation according to claim 14,
wherein the vitamin B is vitamin B₁ or vitamin B₆.

16. The skin external preparation according to claim 10, which further comprises hyaluronic acid or a salt thereof.

17. The skin external preparation according to claim 10, which is a cosmetic or a quasi drug.

18. The skin external preparation according to claim 17, which is a face lotion, an emulsion, a cream, a hair tonic or a pack.
